(19) United States
(12) Patent Application Publication
(10) Pub. No.: US 2024/0335311 A1
(43) Pub. Date: Oct. 10, 2024

(54) MAPPING RESTING-STATE EEG ONTO MOTOR IMAGERY EEG SIGNALS VIA DATA CLUSTERING FOR REDUCED CLASSIFIER TRAINING REQUIREMENTS

(71) Applicant: Kennesaw State University Research And Service Foundation, Inc., Kennesaw, GA (US)

(72) Inventors: Mohammad Yousef Mousa Naser, Marietta, GA (US); Sylvia Bhattacharya, Kennesaw, GA (US)

(73) Assignee: Kennesaw State University Research And Service Foundation, Inc., Kennesaw, GA (US)

(21) Appl. No.: 18/629,156

(22) Filed: Apr. 8, 2024

Related U.S. Application Data
(60) Provisional application No. 63/494,861, filed on Apr. 7, 2023.

(51) Int. Cl. A61F 4/00; A61B 5/372; A61B 5/398; A61G 5/04; G06N 3/0455; G06N 3/096

(52) U.S. Cl. CPC A61F 4/00; A61B 5/372; A61B 5/398; A61G 5/04; G06N 3/0455; G06N 3/096

(57) ABSTRACT

The use of brain signals in controlling wheelchairs is a promising solution for many disabled individuals, specifically those who are suffering from motor neuron disease affecting the proper functioning of their motor units. Almost two decades since the first work, the applicability of EEG-driven wheelchairs is still limited to laboratory environments. In this work, a systematic review study has been conducted to identify the state-of-the-art and the different models adopted in the literature. Furthermore, a strong emphasis is devoted to introducing the challenges impeding a broad use of the technology as well as the latest research trends in each of those areas.

### RELATED APPLICATIONS

This application claims priority to U.S. Provisional Application Ser. No. 63/494,861, filed Apr. 7, 2023, which is incorporated herein by reference in its entirety for any and all purposes.

### BACKGROUND

Over the past several years, Brain-Computer Interface (BCI) applications have attracted more and more researchers, taking advantage of the advancements in computational capabilities and Machine Learning (ML) science. BCI refers to a system designed to interact with the brain to extract a certain level of valuable information that reflects its complex functions for use in engineering and medical applications.

However, improvements are needed.

### INCORPORATION BY REFERENCE

Citation or identification of any document in this disclosure is not an admission that such document is available as prior art to the present disclosure.

### SUMMARY

Electroencephalography (EEG) is a non-invasive technique for detecting brain activity. Due to its low cost and simplicity, it is a common neural acquisition technique in the Brain-Computer Interaction (BCI) field. EEG is a term for any brain signals detected by an EEG headset. The mechanisms by which those signals are generated are many. Motor Imagery (MI) is one of those methods where the EEG signals are triggered by performing imaginative physical movements of one of the body limbs. The present disclosure relates to using MI to develop BCI-controlled devices including but not limited to assistive technologies like wheelchairs and prostheses. MI is capable of being used in lab environments.

MI paradigm implementation in real-life scenarios may be limited due to its variability among different users. As such, MI may be a subject-specific neural paradigm. This paradigm requires conducting a data collection session, called a classifier training stage, before using the MI-powered tool. During a session, a subject's EEG signals are recorded while performing one or more MI tasks. Data is analyzed and processed after the session to construct a classification model for a subject. A schematic diagram of an exemplary MI application workflow is shown in FIG. 1.

Use of a training session may be a drawback of the paradigm. The present disclosure thus relates to methods of minimizing the duration and the extensiveness of data collection sessions. Transfer Learning (TL) methods are one such method where TL is capable of transferring knowledge between different users. With a large pool of users, some knowledge of neural behavior can be adjusted and reused by another user, shortening the duration of the training phase.

The present disclosure relates to an additional method to minimize training requirements including use of rest EEG signals. Rest EEG signals are defined herein as the signals recorded when no intentional mental tasks are performed by the user. Rest EEG behavior carries a wealth of information about the user; therefore, it can be utilized to minimize the MI training requirements. Thus, the present disclosure relates to drawing correlations between rest EEG signals and MI EEG signals, demonstrating the soundness of exploring minimizing or eliminating the training sessions by utilizing resting EEG state signals.

Brain-computer interaction (BCI) implementation is represents a straightforward process. Achieving adequate performance for real-life applications using BCI represents a challenge. Thus, there is a need to carry BCI field from the research domain to solve real-life problems. Hence, work is still needed to exploit the technology for the wide range of applications BCI can serve. The present disclosure thus relates to BCI in engineering applications, as disclosed herein.

### BCI in Biomedical Engineering Applications.

One use of BCI is controlling a screen cursor to help individuals with limited physical abilities interact with computers without asking for external help. Due to the simplicity of the task, it was one of the first BCI applications to receive attention from the scientific community. Such a use of BCI involves few commands needed from the subject and lax timing requirements. The application is still arguably limited in its capabilities and lacks smoothness for broad implementation. Hence, this application needs improvement. Some research has succeeded in achieving satisfactory performance from an accuracy perspective (upper 90s). Nevertheless, the technology suffers from a number of issues, such as extra hardware needed to facilitate the system parts (higher cost) or the personalized nature of the model (designed for individual users).

Another BCI use is in the restorative technology field including the development of modern, intelligent artificial limbs (prostheses). Limbs may be complex due to the high number of tasks the artificial limb must be able to perform to imitate the physiological organ. Prostheses are often constructed using multiple modalities including but not limited to Electroencephalography (EEG) and Electromyography (EMG), or combined EEG paradigms. Implementation of BCI-based prostheses is still narrow. Therefore, improvements of such prostheses are required.

The present disclosure relates to BCI-driven wheelchairs operated by human brain signals. An additional use of BCI is the design of BCI-driven wheelchairs that are operated by human brain signals. This may help individuals that rely on wheelchairs to meet their movement needs. As a non-limiting example, it may help those diagnosed with any type of Motor Neuron Disease (MND). MND is a medical condition that restrains the person's physical motion ability while maintaining a fully functional brain. Symptoms of MND begin to appear gradually and keep developing with time till all skeletal muscle activities become impossible. One of the common types of MND is Amyotrophic Lateral Sclerosis (ALS). Additionally, BCI wheelchairs can be beneficial for individuals diagnosed with any medical condition affecting mobility, such as Spinal Cord Injury (SCI). Every year, around 5000 people are diagnosed with ALS and there were approximately 294,000 active SCI patients in 2020 in the U.S. alone. With 13.7% of the adult population in the U.S. living with a mobility disability in 2020, smart wheelchairs have the potential to improve the quality of life of many people around the globe. Thus, improvements in this field are needed.

### Neural Activity Measuring Techniques

Brain activity can be explored using different techniques. BCI's different techniques may be considered based on invasiveness. Invasive techniques require surgical intervention for the placement of certain tools inside the human body. These tools include but are not limited to microchips. Though this technique offers direct interaction with the brain, it requires human resources including but not limited to time, effort, and human skills, which translates into higher costs. As a result, it is less researched compared to non-invasive techniques that do not require such resources.

EEG is a non-invasive technique used in BCI. EEG includes low cost and an increased overall effectiveness in depicting brain activity. It may include a headset of electrodes that provides a low-resistance path to the electrical signals generated from the underneath brain activity. These signals are then processed. Such processing identifies brain activity associated with signal behavior. Several challenges pertain to EEG. Several non-limiting examples of challenges are provided below. EEG signals are naturally poor in spatial resolution, in order of a few centimeters. This is mainly due to the way the signals are transferred from the bottom layers of the brain (the source) through the brain tissues to the scalp where the electrodes are placed. This is called the Volume Conduction (VC) effect. VC creates a challenge of mapping the electrodes to the targeted brain region, which already has little known about its functional structure. The temporal resolution of EEG is considerably better, in order of milliseconds. The nonstationary behavior of EEG causes the technique to be more susceptible to environmental conditions such as surrounding noise, and makes it prone to artifacts including but not limited to eye movement and biological processes inside the body, leading to an overall low Signal-to-Noise-Ratio (SNR). As a result, EEG resembles a signal processing challenge to properly utilize the non-linear distorted brain signals.

Besides EEG, functional Near-Infrared Spectroscopy (fNIRS) is an additional technique for characterizing brain activity. It is based on a different methodology than EEG. It detects neural activity by sending an infrared light into the head tissues. This light gets absorbed by the oxyhemoglobin and deoxyhemoglobin and based on this absorption, blood oxygenation levels are estimated and these reflect the neural activity at the cortical location. fNIRS can be portable, easier to implement, and less susceptible to artifacts compared to EEG, but it can be more expensive. The two techniques can be coupled together for enhanced performance. In addition, medical imaging techniques can be applied with EEG and fNIRS, but those are rarely used for engineering applications due to their complexity and high cost.

### BCI-Driven Wheelchairs

BCI-wheelchair applications may include higher risks associated with system malfunctions. This application is time-sensitive. The right command must be issued at the right time for proper operation. Though less significant in other applications, for wheelchairs, this may lead to serious injuries. Also, BCI wheelchairs are expected to operate in a dynamic environment with several variations in the surrounding objects every time a task is performed. These challenges are specific to the wheelchair application. EEG-powered wheelchairs may also exhibit challenges.

The Motor Imagery (MI) type of Electroencephalography (EEG) signals are triggered by imagining physical movements by body limbs. The EEG signal characteristics generated by each mental imagery of a limb can be decoded using machine learning classifiers to determine the corresponding task performed by the user. Classifiers undergo pre-training using a dictionary of EEG signal types that are associated with MI tasks. By doing so, the classifier is able to learn how to map new EEG signals to the MI task executed by the user. This information can then be utilized to issue control commands to external devices.

The present disclosure relates to eliminating the need for training that limits any MI application to specific users that performed the training session composed of performing several cycles of MI tasks. This is done by the one-to-many mapping scheme outlined by the following procedure.
1) From data available online and generated from large pool of users, extract:
   a) MI task-EEG signals dictionary (many);
   b) Rest-EEG signals (single);
2) For (a), generate a confusion matrix that represents the contrast between the EEG signals associated with different MI tasks for each user in the pool.
3) Cluster the resulting matrices in (2) based on the values of rest-EEG signals (b) (approach demonstrated by our preliminary results).
4) Get a representative confusion matrix for (3) that adequately describe the distinction between MI tasks for all users in each subcategory.
5) For a potential user, collect short segment of rest-EEG in real time. Then, find the subcategory where this EEG segment belongs. Then, use the representative confusion matrix for that subcategory from (4).

The present disclosure relates to replacing the need for pre-recorded many tasks data collection by a single recording obtained in real-time. This is done by mapping those many tasks to a representative distinction matrix obtained from a single recording.

This approach is different from available patents and literature. Those who are utilizing rest-EEG (such as (CN111067517)) are only doing it to eliminate people who cannot perform satisfactory distinction between the MI classes. The present disclosure relates to a much simpler and more streamlined problem. The prior art yields performing classification based on rest-EEG, while others are only concerned with identifying those with very low classification results. The present disclosure also differs from applying Transfer Learning to shorten training time (such as (CN111582082)). Transfer Learning is the process of borrowing MI data from other subjects to minimize the training needs for a new subject. In this model, training is still required and never eliminated as no user-specific data is generated to match the new user's data to the data from the pool. In contrast, the present disclosure eliminates training and replaces it with an obtainable real-time segment, which is not used for training the model. Instead, the model is already trained and can be sold in an off-the-shelf fashion, as the necessary data for matching is generated after the model has been created and sold to potential customers.

### BRIEF DESCRIPTION OF THE DRAWINGS

The following drawings show generally, by way of example, but not by way of limitation, various examples discussed in the present disclosure. In the drawings:

FIG. 1 shows a diagram of an exemplary motor imagery (MI) workflow according to the present disclosure.

FIG. 2 shows an original protocol and additional protocols according to the present disclosure.

FIG. 3 shows a representation of brain regions according to the present disclosure.

FIG. 4 shows a diagram of exemplary signal processing steps according to the present disclosure.

FIG. 5 shows an exemplary scheme of electroencephalography-driven (EEG-driven) wheelchairs according to the present disclosure.

FIG. 6 shows exemplary stages of Preferred Reporting Items for Systematic review and Meta-Analysis (PRISMA) according to the present disclosure.

FIG. 7 shows the number of Brain-Computer Interaction (BCI) and Electroencephalography (EEG) publications according to the present disclosure.

FIG. 8 shows a conceptual diagram of methods according to the present disclosure.

FIG. 9 shows experiment paradigms for three datasets used according to the present disclosure.

FIG. 10 shows data on quantifying motor imagery (MI) characteristics from rest according to the present disclosure.

FIG. 11 shows a node freezing-transfer learning (NF-TL) method according to the present disclosure.

FIG. 12 shows an experimental protocol for data collection according to the present disclosure.

FIG. 13 shows example MI distributions for 3 subjects (S1, S2, S3) belonging to a single Rest group.

FIG. 14 shows a graph demonstrating accuracies achieved via constructed and original models.

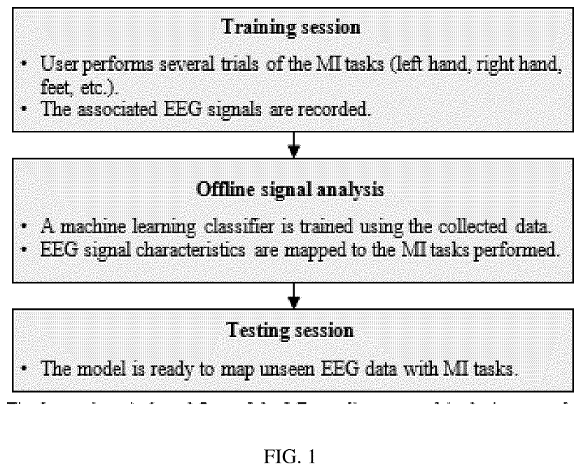

### DETAILED DESCRIPTION

The use of brain signals in controlling wheelchairs is a promising solution for many disabled individuals, specifically those who are suffering from motor neuron disease affecting the proper functioning of their motor units. Almost two decades since the first work, the applicability of electroencephalography-driven (EEG-driven) wheelchairs is still limited to laboratory environments. The present disclosure relates to improvements over the prior art.

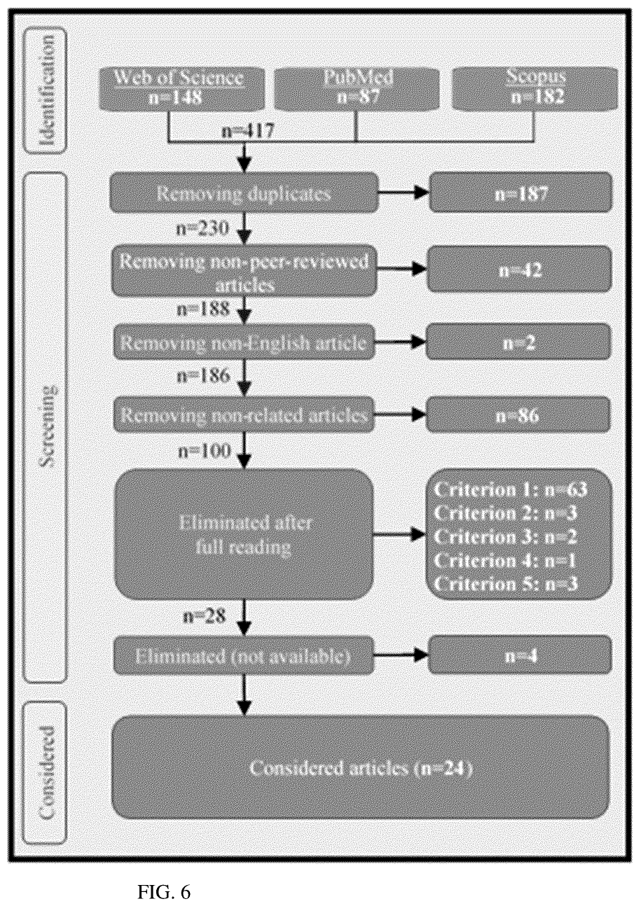

Millan et. al. performed research on brain-computer interaction (BCI) for mobility applications. The present disclosure relates to implementations of BCI wheelchairs. The search was conducted in accordance with the Preferred Reporting Items for Systematic review and Meta-Analysis-Protocols (PRISMA-P as shown in FIG. 6) guideline where the search and consideration criteria are documented for better reproducibility.

### Search Strategy and Eligibility Criteria

The present disclosure relates to a search using Web of Science, PubMed, and Scopus. IEEE was excluded as it is part of Scopus. The keywords and the Boolean expressions used, along with the applied filters, are shown in Appendix A. The keywords were chosen to obtain the results specifically related to EEG-driven wheelchairs. Also, the search was limited to peer-reviewed articles written in English. These filters were only available in Web of Science and Scopus. The search was also limited to the article's title and abstract to limit the number of studies acquired. This is a plausible course of action, assuming the articles related to designing BCI wheelchairs would include the keywords BCI, EEG, and wheelchair in their abstracts.

The procedure followed in obtaining the studies considered herein is shown in Appendix A. The total number of articles retrieved was 417. The papers were exported and downloaded from the three databases in Comma Separated Value (CSV) format. Then, the duplicated articles were removed by Excel using the Digital Object Identifier (DOI) of each. Papers without DOI were removed manually. Next, all manuscripts that were not peer-reviewed or written in any language other than English were removed. These were obtained from PubMed where no filters were available in the search engine (see Appendix A). Subsequently, the articles that were deemed irrelevant were removed, mainly violating the search guidelines by not being a technical article addressing EEG-driven wheelchairs. Some such articles studied a specific aspect of EEG-BCI applications, such as new feature extraction or classification techniques. This type of article mentioned the term “wheelchair” in the abstract as a direct BCI application. After that, the rest of the articles went through a reading step to determine which articles to exclude according to the following five rejection criteria:

Criterion 1: No real-life navigation test using a full-size wheelchair. This is to account for any differences in performance resulting from user fatigue or any variables introduced by the environment or the hardware design. Hence, studies consisting of virtual (simulated) wheelchairs or small robots in place of wheelchairs were not included.

Criterion 2: The EEG paradigm or acquisition details are not given. These are the articles where significant details about the paradigm used are missing. Such missing details include the EEG acquisition procedure, testing environment, etc.

Criterion 3: No results reported.

Criterion 4: Unclear testing procedure or results.

Criterion 5: Wheelchair results embedded with other functions.

The five exclusion criteria were applied to keep focus on the practicality aspect of BCI wheelchairs. The insightful papers that were excluded may still be used to explain some methodologies but will not be part of the comparison carried out later. Of what is left (28 articles), 4 papers were not available, 3 because the journal repository is not accessible anymore, and the last one because it has been retracted. The final count for the articles considered is 24. Every step of the literature review process was repeated twice to ensure accuracy.

### Considered Articles

The articles resulting from the search are shown in Table I sorted by the EEG paradigm used. Details and performance metrics are included in the table as well. The present disclosure below relates to the needed background information to comprehend the information in the table. The performance metrics will be compared in the following section.

### Observations

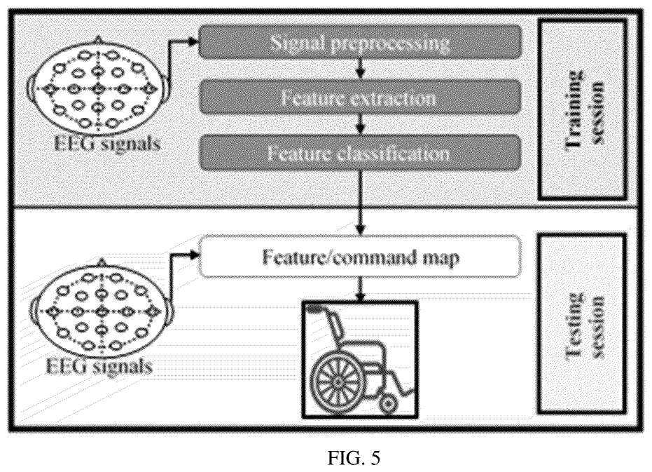

EEG is a modality used in smart wheelchairs. This is mainly due to the large amount of information that can be extracted from the brain due to its complex and essential role in human cognitive and physical activities, and because it does not require any physical activity from the human, which is a significant advantage of EEG over EMG, Electrooculogram (EOG), and eye tracking for this type of applications. EMG, EOG, and eye tracking have been used in conjugation with EEG to leverage the special characteristics of each. A simplified scheme of the EEG modality as applied in wheelchair control is shown in FIG. 5. More details on the steps in the figure are provided below.

### EEG Paradigms

EEG signals can be categorized into two groups based on how they are triggered: spontaneous and evoked. While evoked signals only arise when triggered by external stimuli, spontaneous signals are self-generated signals that reflect the human mental and awareness status. Motor Imagery (MI) signals belong to the spontaneous type of signals. They appear on the EEG as a result of mentally performing motor tasks using several body parts, such as the hands, the feet, and the tongue. MI signals are identified by a decrease in brain activity before and while performing motor tasks in a process known as Event-Related Desynchronization (ERD). After the motor task is completed, whether physically or mentally performed, the brain activity returns to normal in a process called Event-Related Synchronization (ERS) [35]. There are spatial differences related to ERD and ERS as well. The tempo spatial characteristics of ERD and ERS depend on the limb used and on the rhythm of interest. MI is mostly associated with two brain rhythms, Beta (B) and Mu (u). For hand MI, it is been shown that for ERD, a dominant neural activity exists over the contralateral side of the brain (opposite to the side of the limb used), while ERS shows high activity on the ipsilateral side of the brain, and this is valid for both the ß and u rhythms but it is less evident for B-ERS.

For evoked signals, two types are capable of being used in BCI: P300 and Steady-State Visual Evoked Potential (SSVEP). P300 is a signal that appears approximately 300 ms after the onset of an uncommon stimulus, whether it is visual, auditory, or somatosensory. This type of signals belongs to a group of signals called Event-Related Potential (ERP), which represents the signal behavior in the brain after experiencing an infrequent stimulus. This paradigm is used in BCI by utilizing different stimuli that each flickers with a slight shift from the other. This is to allow linking the signal observed on the EEG with the stimulus chosen by the user. For example, one stimulus can flicker at t₀ and every other stimulus n can set to flicker at tₙ+t₀. The other evoked signal paradigm, SSVEP, is triggered by lighting stimuli. These signals, unlike P300, are periodic, following the periodic stimulus rather than a single spike. The frequencies of the generated signals are integer multiples of the stimulus' fundamental frequency. Hence, a high number of commands are achievable by using stimuli flickering at distinctive low frequencies.

Both signal types, spontaneous and evoked, each hold a different set of pros and cons. For evoked signals, several cycles are needed to conclude the final decision. This is to ensure robust translation of the response signal. In other words, to mitigate possible noise and artifacts from the environment. This issue is critical as it slows down the command translation process. Also, evoked signals can cause tiring for the eye due to the continuous exposure to flickering sources. While this issue does not exist in the MI paradigm, MI suffers from a higher mental workload placed on the brain compared to evoked signals; both P300 and SSVEP do not require performing mental imagery tasks. One advantage of MI is that it gives the user a full sense of control as the user can issue a command at any time, unlike the evoked signals case where a sequence of stimuli must occur to issue the command. Because of that, MI systems may be called active systems, and evoked signals are called reactive systems. A disadvantage of active systems is the need for user-specific training sessions before the chair is used. This is a result of the non-stationary and noisy nature of EEG signals that cause variability in response among subjects and across different sessions. This issue necessitates building the classifier using data from the same subject that will be using the classifier in real time at a later stage. An additional disadvantage of MI is the limitation on the number of commands, which arises from the limited number of motor activities possible to perform by the user. One solution disclosed herein is using other biological modalities to perform the commands not achievable by EEG, such as EMG, EOG, and eye movement and position tracking. It is believed that user MI training will result in better performance with time as the user adapts to the classifier by receiving several feedback cycles. P300 on the other side is expected to have lower performance with time because of aging. For the above reasons, both BCI systems, active and reactive, include limitations to be addressed. Both BCI systems also have potential of transferring the technology to the real world. Table II shows a side-by-side comparison of the different paradigms.

The three EEG paradigms (MI, P300, and SSVEP) can be used alone, or in a combination of two or three. Any use of multiple paradigms is referred to as hybrid models in this article. EEG can also be combined with other physiological modalities to form what is known as multi-modal BCI systems. From Table I, secondary modalities include speech, and EOG, which is a technique to measure the surface voltage from the eye, mainly triggered by blinking. In addition to the aforementioned EEG paradigms, certain work used spontaneous EEG signals different from the MI paradigm. Others used the intensity level of the filtered signal in the alpha band. Still others targeted Mental Tasks (MTs) which they are ERS/ERD signals different from MI. Others used math solving, text reading, and relaxing. Mental arithmetic operations and word chains may be used. The use of MTs is less common than MI. From Table I, all paradigms are being used, which indicates that no ideal model is determined. It is believed that MI provides an enhanced user experience compared to other modalities in navigation applications. For example, associating right and left directions with right and left MI is more intuitive than using MTs.

### Control Methodologies

The control level refers to the method of interaction with the chair. The present disclosure relates to two control methodologies, these are low-level (asynchronous) and high-level (synchronous). In the first type, the user controls the wheelchair through directional commands. The EEG signals from the user are mapped to directional chair movement commands, such as right, left, forward, etc. For example, a right-hand MI is mapped to a 90° right turn of the wheelchair. High-level systems on the other hand refer to systems where the EEG signals are mapped to destinations rather than directions. The destinations are built-in (pre-programmed) into the model. So, each EEG signal will select a different destination that the wheelchair is capable of automatically driving to it. There is also a third type of control, which is a combination of low and high-level control commands, known as hybrid systems.

Table I shows systems that adopt the low-level model. These systems offer flexibility in navigating to unseen places and the do not require external aid. Also, few articles show an independent high-level system despite its advantage of requiring little mental work and training from the user. Such a system is limited by the list of pre-defined locations; modifying the list would probably need an intervention by someone besides the user. Another reason is the extra equipment required for environment detection and path planning. This is crucial from a cost perspective as accurate path navigation and collision avoidance systems require fast and reliable environment detection equipment implying higher cost, so it is generally less appealing to the audience.

### Data Acquisition

An exemplary piece of information for BCI systems is the details of the EEG signals acquisition, mainly the number of electrodes and the targeted brain region. This is useful to assess the feasibility of the technique as setting up EEG can be a tedious process that requires a certain level of experience from the examiner. The data acquisition details and the preprocessing steps for some of the articles in Table 1 are shown in Appendix A. The brain signals are extracted using an EEG cap composed of a number of electrodes placed according to a well-established configuration called the 10-20 configuration. While more electrodes may offer greater detail, it entails many practical issues. From Appendix A, the number of EEG electrodes and their locations differ across the studies as they target different brain signals active in different regions of the brain.

The MI signals are active over the motor cortex located in the central lobe close to the front region. This explains the use of those regions in most studies utilizing the MI paradigm. The entire area surrounding the motor cortex is usually targeted to account for the poor spatial resolution and the possible shift in electrode positions during the cap install. The occipital region works well in showing the visual effects on the brain, making it preferred to use in SSVEP applications. The location to detect the P300 signal depends on the stimulus type whether it is visual or auditory.

In addition to the poor spatial resolution of EEG, another fundamental reason for the difference in targeted brain regions in the studies is the lack of a solid understanding of the interconnections between the brain regions and the lack of thorough understanding of the brain functional strategies. Accurate targeting for a brain region lowers the number of electrodes needed, which influences the applicability of EEG in social settings. For instance, the average number of electrodes in the SSVEP case is lower than it is for MI. This is due to the smaller area of concentration.

### Signal Processing

A BCI-driven wheelchair system is composed of two parts, as shown in FIG. 5. The first part is the training session, which is a controlled experiment conducted before the navigation test takes place to extract EEG signals from the users. These signals are then downsized via what is called a feature extraction step for use in building and optimizing the ML classifier in a step known as feature classification. Since the classifier is constructed based on subject-specific data, it is called a subject-specific classifier. The second part is the testing session where the navigation test takes place. In this part, the user initiates the commands and classifier performance is reported. The details of each of the steps are out of the scope of this study but below is a brief description of each. The typical EEG preprocessing steps are:

### Amplification

Amplifying the signal is used to increase the SNR to help differentiate the brain signal from the environment-induced noise and artifacts. Amplification can be either done at the electrode level using active electrodes (an individual amplifier is located next to each electrode) or after the signals from all channels are transmitted to the processing unit. The use of active electrodes is valuable in that it is carried out before any interference from the neighboring electrodes or nearby external sources of noise occurs. Passive amplification, on the other side, is not able to address electrode interference as it is done on the data transmission level. Though lower power values are noticed using passive electrodes, other studies show comparable noise levels for both types. This should not be surprising as the added value is dependent on the interference level and experimental conditions, such as the electrode impedance value. For the studies screened in this work, some used active electrodes. With few exceptions, no significant improvement is observed. Note that the four studies were conducted in an indoor environment with limited noise sources, so the value of using active electrodes is most likely underestimated herein.

### Sampling

Sampling is needed to convert the signal from analog to digital format for easier processing. As EEG is known for its noisy behavior and fast activity, down sampling the analog signal is usually done without a major loss in accuracy. Of the studies in Table I, 21 studies used a sampling frequency between 200 Hz and 256 Hz, so this is a reasonable sampling frequency that maintains good signal quality without necessitating overly burdensome computing capabilities.

### Filtration

Filtration of unwanted frequencies is used for all systems. Depending on the targeted frequency range for the brain signals, the appropriate filters to remove other frequency components are applied. Noise and artifacts of high frequencies are easy to identify and remove since EEG signals are mostly of low frequencies. The general filtering procedure for most of the studies screened is to apply a bandpass filter around the needed frequency range along with a notch filter at 50/60 Hz to remove powerline interference. The bandpass filter usually starts at very slow frequencies larger than 0 to avoid Direct Current (DC) interference and extends to around 30 Hz, this covers a, B, and y rhythms in addition to all possible P300 and SSVEP frequencies. The exact filtration range differs in the literature as it is dependent on the noise sources.

### ML Steps (Feature Extraction and Classification)

### Feature Extraction

Feature extraction is meant to extract the features that best represent the signal qualities to reduce the data size with minimal loss in quality.

From Table I, it is evident how the Common Spatial Pattern (CSP) is used frequently in the field of MI-BCI. CSP is a spatial filtering technique based on joint diagonalization of the spatial patterns to find a transformation matrix for each of the two classes. The first and last few rows of each matrix are used to maximize the variance in the band power between the two classes. This method is dependent on the subject and works with only two classes at a time. Thus, when there are more than two classes, it can be done in a one-versus-all fashion, also known as multi-CSP. There have been modifications to the original algorithm such as the popular Discriminative Filter Bank CSP (DFBCSP) method. Moreover, Band Power (BP) features can be used for MI. As the name implies, BP features are the power of the EEG signals in each frequency band. For MTs, many statistical features can be used. Certain studies used four statistical features: mean absolute values, standard, deviation, line length, and the number of zero crossings.

For SSVEP and P300 the process is less critical and not always necessary as the amount of data is smaller compared to MI and it is easier to identify because of the well-defined brain reaction to both paradigms. For SSVEP, since the frequency of the generated signal is known, Power Spectral Density (PSD) can be easily applied to identify a specific frequency. For P300 on the other side, the timing of the generated signal is what is known to the operator. Thus, the use of a Band Pass Filter (BPF) targeting the expected time frame of the P300 response is a valid technique.

### Feature Classification

From Table I, Support Vector Machines (SVM) and Linear Discriminant Analysis (LDA) are techniques used in the literature. LDA is a linear classifier that performs the separation by constructing hyperplanes that minimize the variance within each class and maximize the distance between the two classes' means. It has various variants, such as Functional LDA (FLDA), and Step-Wise LDA (SWLDA). SVM is a classifier that imitates transformation to a higher dimension for maximized separability via using kernel functions. In EEG, the Radial Basis Function (RBF) kernel is very common to use, as well as the Hybrid Kernel Function (HKF).

Canonical Correlation Analysis (CCA) is used for reactive systems to find the correlation between a reference signal resembling the stimulus and the generated signals. It can also be used to link the response recorded in training to the actual signal generated while testing. Filter Bank CCA (FBCCA) is a modification of CCA that is utilized to detect the harmonics of the source fundamental frequency. CCA is only one method of finding the correlation between two signals. Other methods can be applied effectively to classify both P300 and SSVEP response signals.

### Classifier Training

Classifier training refers to the process of collecting data from the subjects for the purpose of constructing the ML classifier. Classifier training may represent a time-consuming process that requires effort from both the examiner and the user, particularly since training is a subject-specific process. It is difficult to succeed in using pre-trained BCI models, as shown in all studies in Table I.

In the table, the training sessions conducted in each of the studies are shown. The term “trial” refers to the instance where the user performs a mental task, whether it is an actual mental task in the case of MI and MT or perceiving a stimulus in the case of reactive systems. With a few exceptions, it is apparent how many trials may be needed before the experiment takes place. Note that the trials must cover all mental tasks or stimuli presented, which increases the time needed to finish the experiment, especially as this session is repeated for each subject. MI training can last for a few hours, while in reactive systems, it can be carried out in a few minutes.

### Testing and Performance Metrics

The number of subjects who carried the tasks and their disability status are included in the table. The letter “H” represents healthy participants, meaning that no mobility disability was disclosed by the subject. The letter “D” refers to individuals diagnosed with any type of motor disability. There have been some studies that discuss the difference in BCI performance between healthy and disabled individuals. Hence, having several disabled individuals performing the tests adds credibility to the effectiveness of the proposed model. Whether the difference in performance is driven by physiological or behavioral reasons, this was observed in at least two of the studies in Table I. Also, the users are classified if they have any experience of any type with BCI applications. This is to weigh the effect of subject training, which is believed to be a determining factor in the overall performance. The difficulty level of the test along with the number of rounds performed by each subject are included as well. The difficulty level is included to provide a reference meaning to the results reported. While most articles conducted a relatively easy indoor navigation experiment, some performed more challenging ones by placing obstacles in close proximity to the user's navigation path for example. High-difficulty tasks refer to challenging experiments where the test is usually conducted in tight places and require maneuvering skills to avoid collisions.

The last section of the table presents different performance metrics important to assessing the different designs. First, there are the accuracy values, as achieved during the navigation task. Accuracy is defined as the number of correct commands captured by the classifiers with respect to the total number of commands initiated by the user in real-time. A more universal testing performance metric is the time taken to cross a certain distance [s/m]. Simply, it is the speed reciprocal. Except where noted, the distance and time were taken directly from the papers. More results are shown in the last column of the table. These include the number of collisions and the number of commands per unit of time or distance, the time and distance taken with respect to the optimal minimum values, and also the number of commands which is a valuable metric for assessing the mental workload placed on the user.

### Comparison

As discussed above, several differences in the designs make the comparison between the different studies challenging. With that, a detailed look at the different articles in Table I is introduced in this section to help shape some conclusions on the methodologies adopted in the literature.

### Spontaneous Paradigm

All MI studies utilize the low-level BCI model, which relates to the limited possible number of MI tasks. Of the seven studies, only two report accuracy values: 65.50% and 76.92%. Both studies used band power features with an LDA classifier. The data acquisition, training, and navigation tests are also similar as they both are coming from the same group of researchers. Though the reported accuracy exceeds the chance level, 50% and 25%, respectively, they are lower than acceptable for practical applications. The low accuracy values are reflected in a long time needed to finish the experiments. For certain studies, the average of 18.20 s/m is a high value, especially considering the simple navigation task of moving in a short straight line with two stops along the route. The short-distance route along with the needed stops resulted in a high number of commands. Though the accuracy may be better in certain studies, the time/distance metric is higher due to the collision detection mechanism that was automatically activated many times to avoid collisions in the tight corridor used for navigation. In both studies, the authors believe that a main driver of the poor performance was the low accuracy values reported in the calibration session; most participants scored in the 70s and 80s range. This is despite the fact that the users with error exceeding 30% were removed from the navigation experiment and claimed BCI illiterate. BCI illiteracy is a condition where the subject cannot produce mental tasks with accuracy above 70%. A similar system may be proposed but less practical as every command moves the chair to a new point of a grid covering the testing area. This operation is less smooth as it requires an increased number of commands to travel between places (e.g., around one command every ten seconds for the navigation task reported). CSP features may be used to train an LDA classifier, which is found in the literature. The installed collision avoidance system was able to avoid all collisions with a rate of 2.58 collision per minute. Based on the given testing area geometry, the speed reciprocal is expected to be elevated.

In certain studies, 31 active electrodes were used to collect the signals. A CSP-LDA combination is used for users with some level of BCI experience who went through training before performing an indoor path navigation task with one waypoint 15 times. The number of commands turned out high as the task required several turns along the way and possibly related to a few collisions that occurred along the way. On average, the chair needed 13.65 seconds to cross one meter of distance including the stop point. Though this is an elevated number, the authors believe the results of this study are truly reflective of the paradigm used as it was carried out with several subjects in a non-trivial task and several meaningful results are reported.

The work in is the only work to conduct the navigation task in an outdoor environment. This significant in assessing the environmental effect on the overall performance. With noting that the signals were acquired with 24 dry electrodes and a typical SVM classifier is used to process the CSP features, an impressive 5.60 s/m on average was needed to perform an intermediate-level task of moving between objects in an open space. The other results are encouraging as well. The time ratio is obtained by using a fixed 0.2 m/s chair speed as a reference and the distance ratio is the ratio of the distance taken to the lowest distance between the start and end points.

Lastly, the single use of MTs was tested in [43]. The use of a combination of classifiers exceeded the accuracy values obtained from the single classifiers and was able to reach a 90% limit. Analyze the overall system performance by only knowing the Root Mean Square (RMS) position error presents challenges, but this study has shown potential in the combined use of classifiers.

### Evoked Signal Paradigms

[48] and [50] use the SSVEP paradigm for operating the wheelchair. They place several electrodes in the occipital region to capture the signal resulting from the lighting stimuli. High accuracies were achieved compared to MI which is expected for evoked signals. The speed reciprocal is approximated to be in the range between 18 s/m and 40 s/m for [50]. The results are based on four different tasks performed by several individuals ranging between 4 and 13 per task. Based on the elevated number of participants and considering the reduced SSVEP paradigm, it should not be surprising to have this elevated s/m ratio. The same metric in the other study [48] is decent. This is mainly due to the easy navigation task performed. From both articles, it is shown how the number of commands is elevated compared with MI. It is expected to induce a lighter mental workload on the user as these commands resemble gazing at the stimuli and not performing actual mental tasks.

The other study of SSVEP is [49]. The model created for this work can be used in low, high, and hybrid formats. The main paradigm is the low-level paradigm where the user initiates control commands in the form of directions to the chair. While traveling using that paradigm, the user is able to program a specific location to be saved for later use in a high-level fashion. The test conducted is a hybrid test composed of two tasks, one carried in low and one in high level. Such a system solves a challenge of high-level systems, which is the need for external aid to enter new destinations.

The results using the P300 model are expected to be comparable to those of SSVEP. The work in [53] achieved a satisfactory performance when using P300 in a low-level fashion for a challenging task (1.12 distance ratio, and 2.64 time ratio). Accuracy (82.5%) is within expected as well.

The work of [54] shows a high-level system utilizing the P300 response. All commands were translated successfully (100% accuracy), and this is expected because of the limited number of commands needed in high-level systems. On average, 15 seconds were needed to initiate the command from the user. Once initiated, the wheelchair automatically drives to the pre-defined destination.

A hybrid model may be used where the user initiates low-level commands to choose a destination of the ones shown on the monitor. This system was useful for short-distanced destinations. This control scheme has resulted in the system being slower than expected. This raises a question on the feasibility of using high-level commands and whether it is easier to navigate to destinations within sight using directional commands. The results in the table are obtained by applying the low-level scheme. The accuracy achieved is more than satisfactory, especially when noting that the participants were a mix of healthy and disabled individuals. The signals are obtained using active electrodes as well. A low number of commands results from a large portion of the route is a straight path.

### Hybrid Models

Hybrid models refer to models of more than one EEG paradigm. Certain studies both utilize combining the MI and P300 paradigms to navigate the wheelchair by directional commands. Both studies use the CSP-LDA combination and conduct relatively challenging navigation tasks in tight corridors. Both also provide satisfactory results, but neither noticeably outperforms other work in the literature. The use of multiple paradigms may offer new capabilities that are not necessarily reflected in the results. One, non-limiting example of this is the use of the evoked signal paradigm to control the chair speed while dedicating MI for issuing the navigation commands, such as in [59]. Another possible use is for enhanced decision robustness.

Combining MI with SSVEP for low-level systems may be tested. For a challenging task, the rate of collisions is one per forty meters, which can be seen as acceptable considering the difficulty of the task. As well, it is worth noting that high classification accuracy values were achieved in the study (above 90%) for both MI and SSVEP.

Certain results show comparable performance obtained by conducting simple navigation tasks. The metrics reported do not show a major increase in performance compared to some of the single paradigm articles in the table.

### Multi-Modal Models

Multi-modal models refer to models of more than one modality. Here, EEG is one of the two. One may extract the signals using a headband sensor rather than an EEG cap. While this is a more practical approach, it is limited by the area it covers. In this work, the intensity of the a rhythm as well as eye blinking signals appearing on EEG are taken as features. This approach resulted in an elevated s/m metric for a simple navigation task.

Human speech may be used to initiate the stop command, P300 to start the chair, and MI for the navigation commands. The test is a very simple one in a half-circle path carried out ten times per each of the five subjects. The s/m achieved is the lowest one seen in the literature. This positive result is further confirmed by the optimal time and distance ratios. Note that this performance is compromised by the technical challenges of facilitating the different modalities used.

Another common modality to use with EEG is EOG. It is considered a different modality in this study because it requires a different action besides mental activity. MI may be combined with EOG. The number of collisions per meter is around 1/30m. P300 may be combined with EOG. It is applied in both low and high-level fashion. Both were separately tested. It is clear that the low-level system is slower because of the higher number of commands needed. Note that both results are satisfactory compared to the other studies in the table. Lastly, both P300 and MI may be combined. A challenging task was conducted by four experienced subjects, and satisfactory results were achieved.

Additional challenges of EEG-driven wheelchairs that prohibit a wider use of the technology are introduced below.

### Lack of Standardized Testing

EEG-driven wheelchairs have many variables contributing to their final performance. To that end, a unified strategy to test the final product is a tricky task, and a single metric would not be sufficient to describe all details of the system. The metrics used in the literature are introduced in Table III.

While accuracy, is a metric used in a lot of BCI applications, it has limited significance in high-level/hybrid systems as the number of commands issued in such systems is limited. Thus, the time/distance metric is a better representation of system performance. This metric is not commonly used in the literature (the values reported in Table I were calculated by the authors). The metric though does not adequately account for any stop points along the route or the difficulty level of the navigation path. Additionally, the ratios of actual time and/or distance with respect to the minimum values help assess how close the developed model is compared to regular wheelchairs. The number of collisions is used to ensure system safety. In short, there are several metrics to report. Excluding some of them may produce incorrect or faulty conclusions. It may be suggested to report all metrics in Table III to avoid inaccurate conclusions. If these are reported, a good indication of the overall performance is possible, regardless of the navigation task.

The mental workload of the paradigm is another critical factor to consider. The direct way to assess the workload is by observing the number, duration, and type of tasks performed. Some researchers prefer using the well-established NASA task load index, while others come up with their own assessments. The use of surveys is also possible. And lastly, a more systematic signal-based way is possible by studying the brain activity by means of ML.

Issues with the MI Paradigm

Due to advantages of MI over reactive systems, it is believed to have a real potential to become applicable in real-life settings. One limitation of MI is the training preceding the experiment. Longer training sessions would generate better results. Classifier training is not only critical as it increases the requirements of the model, but it acts as a major barrier to commercial BCI wheelchairs since the user needs to train the model and cannot obtain it pre-programmed.

An additional limitation with training is that it should be carried out for each subject. This is a tedious step for potential disabled users. This becomes more concerning knowing that the motor neuron abilities of MND patients keep changing with time. In addition, there have been several studies that show age as a responsible factor for changing the brain signals behavior with time. All of this indicates the instability of current approaches. To that end, the authors believe that adopting dynamic MI models where less user-specific data is required will boost the applicability of such systems. A dynamic classifier is a classifier that needs a minimal amount of information from the user and keeps adjusting its parameters based on data obtained in real-time. This will eliminate the training session and results in a classifier more adaptable to different subjects and conditions. Reducing the training time is significant and extends beyond wheelchairs to several BCI applications. In the studies, the concept of transfer learning was deployed where only a small data set is needed from the targeted user and promising results of equal and even exceeding other user-specific training models were achieved, which shows a positive trend towards building robust universal classifiers.

An additional limitation is the small number of achievable commands with adequate accuracy. Most researchers target the use of up to three MI movements, these are both hands and feet. If a wheelchair design requires a higher number of commands, these can be achieved by another modality or EEG paradigm.

Issues with Evoked Signals Paradigms

One of the challenges of P300 and SSVEP is the non-intuitive way the chair is being controlled which may cause hesitancy from potential users to use the technology. Also, these systems may require extra equipment to be mounted on the chair to facilitate having the stimulus. As well, since these paradigms may be applied in a high-level fashion, they require more expensive equipment for path planning and obstacle avoidance. Additionally, the long response time in these models may be difficult to overcome as it is an artifact of the design itself.

Another issue for P300 models is the possibility of reduced performance with time as the eye gets used to the stimulus. Hence, the stimulus needs to be periodically changed. More crucially, both P300 and SSVEP may be tiring to the eye, but the extent of this issue is still largely unexplored and needs to be addressed.

### EEG Impracticality

A challenge of EEG-based BCI is its reduced practicality due to potential accidents. First, the use of a large array of electrodes increases the risk of individual electrode failure. Second, if wet electrodes are used, this requires frequent gel filling, which is a time-consuming process and cannot typically be done by the disabled individual. Also, it can be challenging to effectively attach the cap to the user's scalp and establish a secure connection, this is often the case for users with thick hair. These reasons make it more difficult to apply EEG in normal life situations and make it unappealing in social settings. In recent years, the industry started introducing modern easy-to-set-up EEG headsets. Although these are limited in their spatial coverage, they prove the possibility of building light and easy-to-use EEG headsets, especially if the targeted brain region is small. Thus, a step towards easier use of EEG would be examining the possibility of reducing the number of electrodes by fully capturing the brain activity by a selected number of electrodes. This is dependent on an understanding of the functional behavior of the brain. If fully understanding the brain functions is a lengthy and challenging process, then applying ML concepts to utilize the number and location of EEG electrodes would be a cheaper, easier alternative. Several studies have shown the validity of this approach.

An additional issue of EEG is the low signal-to-noise ratio (SNR), which makes distinguishing the useful signal a challenging task, especially in a highly noisy environment. The unpredictable noise sources in the outdoor environment make it more difficult for this application. All studies in Table I, except for one, were conducted indoors with limited noise artifacts. Noise sources include lighting, motion, and any surrounding electrical devices. Some other noise sources are internal, generated from the biological systems inside the human body such as the hemodynamic continuous operation. These artifacts and noise sources can be easily removed when a great difference exists between their range of operation and the typical frequencies of brain signals. Successful transition of EEG to the outside environment may require a robust filtration method.

This review examined 24 peer-reviewed articles related to EEG-driven wheelchairs, with an emphasis on the feasibility of the current models. This article detailed the different approaches available in the literature, as well as the background material needed to understand the theory underlying the technology. Differences among the models leave challenges to be addressed for the strategies moving forward. The article highlighted the pros and cons of the different systems and the needed improvements in the fields of data science, neuroscience, and signal processing to overcome the current challenges for wider implementation of the technology.

Although PRISMA guidelines were followed in conducting the review, human error and search strategy errors in data collection and/or interpretation may serve as a limitation.

TABLE I
STUDIES CONSIDERED IN THIS WORK.

| | | | | | | EEG | Navigation test |
|---|---|---|---|---|---|---|---|
| | | | | | | classifier | Subjects^{1,#} |
| | | Control | Work, | MI/MT | | Training/ | Tot. |
| Paradigm | | level* | yr. | features | Classifier | sub.*** | num. |
| Spontaneous | MI & MT | L | [41], 2018 | BP | LDA | 160 trials² | 10H |
| | MI | L | [42], 2018 | CSP | LDA | 270 trials, | 7H |
| | | | | | | subject to user | |
| | | | | | | performance² | |
| | MT | L⁵ | [43], 2017 | Statistical | ANN, | 30 trials | 3H |
| | | | | features | SVM & RF | | |
| | | | | and BP | | | |
| | MI | L⁵ | [44], 2017 | BP | LDA | 160 trials² | 9H |
| | MI | L | [45], 2017 | CSP | SVM | — | 4H |
| | MI | L⁵ | [46], 2013 | CSP | LDA | 120 trials | 1 |
| | MI | L⁵ | [47], 2011 | BP | LDA | 360 trials | 2 |
| SSVEP | | L⁵ | [48], 2021 | — | CCA | 160 trials | 8 |
| | | L, H & | [49], 2020 | — | PSDA | —² | 5 |
| | | B^{5,7,8} | | | | | |
| | | L | [50], 2013 | — | Statistical | 80 sec session² | 13 (12H) |
| | | | | | maximum | | |
| P300 | | L, B^{9,10} | [51], 2021 | — | LDA | 81 trials | 13 (7H) |
| | | B^{11,5} | [52], 2019 | — | LDA | 144 trials | 5H |
| | | L⁵ | [53], 2013 | — | Bayesian | 3 min session | 11 (10H) |
| | | | | | classifier | | |
| | | H⁵ | [54], 2010 | — | SVM | 716 trials | 5H |
| Hybrid | MI& | L | [55], 2022 | CSP | MI: HKF-RVM | MI: 160 trials^{2,13} | 8H |
| | SSVEP | | | | SSVEP: CCA | SSVEP: — | |
| | MI& | L | [56], 2017 | CSP | LDA | MI: 540 trials | 8H |
| | P300 | | | | | P300: 2700 trials | |
| | MI& | L | [57], 2014 | CSP | SVM | MI: 120 trials | 3H |
| | SSVEP | | | | | SSVEP: 3 min | |
| | | | | | | session | |
| | | | | | | Hybrid: 168 trials | |

| | | Navigation test | | | | | |
|---|---|---|---|---|---|---|---|
| | | | | | Performance metric | | |
| | | Subjects^{1,#} | Test | | | Time/ | |
| | | BCI | Diff. | Rounds/ | Acc. | dist. | |
| Paradigm | | exp. | Level** | sub¹ | [%] | [s/m] | Other |
| Spontaneous | MI & MT | 1 | L | 2 | 65.50 | 18.20³ | 1.48 comm./m |
| | MI | 7 | I | 15 | — | 13.65⁴ | 0.02 coll./m |
| | | | | | | | 0.11 coll./min |
| | | | | | | | 2.25 comm./m |
| | | | | | | | 9.88 comm./min |
| | MT | — | L | 1 | — | — | 0.37 m RMS |
| | | | | | | | position error |
| | MI | 1 | L | 1 | 76.92 | 60.65 | 1.53 time ratio |
| | MI | — | L⁶ | 10 | — | 5.60 | 0 coll. |
| | | | | | | | 1.05 dist. ratio |
| | | | | | | | 1.17 time ratio |
| | MI | — | L | 9 | — | — | 2.58 coll./min |
| | | | | | | | 6.61 comm./min |
| | MI | 2 | L | 4 | — | — | 0 coll. |
| | | | | | | | 4.98 comm./min |
| SSVEP | | — | L | 1 | 93.90 | 8.13 | 1.8 comm./m |
| | | | | | | | 13.24 comm./min |
| | | — | I | 1 | 89.87 | — | 0.93 comm./min |
| | | 0 | I | 1 | 96.23 | — | 21.98 comm./min |
| P300 | | 1H | H | 1 | 94.75 | — | 1.48 comm./min |
| | | — | L | 1 | 79.40 | 23.55¹² | — |
| | | 5H, | H | 1 | 82.50 | 11.71 | 0 coll. |
| | | 1D | | | | | 1.12 dist. ratio |
| | | | | | | | 2.64 time ratio |
| | | — | L | 1 | 100 | — | 15 s response time |
| Hybrid | MI& | 3 | H | 1 | — | — | 0.025 coll./m |
| | SSVEP | | | | | | |
| | MI& | 5 | I | 6 | — | 14.84 | 1.47 comm./m |
| | P300 | | | | | | 5.81 comm./min |
| | | | | | | | 0.05 coll./m |
| | | | | | | | 0.27 coll./min |
| | MI& | 3 | L | 2 | — | 14.60¹² | 0 coll. |
| | SSVEP | | | | | | |

¹Highest number is reported;
²Certain threshold to qualify for navigation test;
³Includes two stops;
⁴Includes one stop;
⁵Equipped with environment detection system;
⁶Carried in outdoor environment;
⁷Destinations saved when passing by it;
⁸Reported results are for hybrid system;
⁹Low-level commands to choose destinations;
¹⁰Reported results are for the low-level system;
¹¹Destinations within sight;
¹²Approximated distance;
¹³Extra session to determine thresholds (MI and SSVEP);
*L—low, H—high, B—hybrid;
**L—low, H—high, I—intermediate

TABLE II
DIFFERENT PARADIGMS USED FOR
EEG-DRIVEN WHEELCHAIRS.

| | EEG signals | | |
|---|---|---|---|
| | Spontaneous | Evoked | |
| Types | Self-generated | Evoked by external stimulus | |
| Paradigm | ERS/ERD(SMR): MI | ERP: P300 | VEP: SSVEP |
| | Generated by | Generated 300 | Generated after |
| | executing | ms after | experiencing |
| | mental motor | experiencing | a flickering |
| | movements | an infrequent | light source |
| | | stimulus | |
| Method | Every motor task | The timing of | The frequency |
| | is associated | the generated | of the |
| | with a | signal is | generated |
| | different | linked to the | signal is |
| | command | stimulus | linked to |
| | | | the stimulus |

| | | |
|---|---|---|
| Eye fatigue | Lower | Higher |
| Mental | Higher | Lower |
| workload | | |
| User training | Extensive | Minimal |
| Num. of | Limited | Higher |
| commands | | |
| Setup | Low | Higher |
| requirements | | |
| Control | Full self-control | Pseudo self-control |
| | (natural driving | (dependent on stimuli) |
| | feel) | |
| Performance | Expected to | Drops for P300 with |
| with time | increase with | aging [40] |
| | user training | |

TABLE III

| Metric | Indication | Notes |
|---|---|---|
| Accuracy | Classifier | Less indicative for |
| | performance | high-level and |
| | | hybrid control s |
| | | ystems |
| Time/distance | Overall general | Unsuitable to |
| | performance | handle stops |
| Normalized num | Mental workload | Commands have a |
| of commands | | different meaning |
| | | based on the |
| | | paradigm used |
| Normalized num | Control system | The presence of |
| of collisions | effectiveness | control system |
| | | should be |
| | | disclosed |
| Training time | Generalizability | Less critical for |
| | and classifier | reactive systems |
| | rigidness | |
| Time ratio | Overall general | — |
| | performance | |
| Distance ratio | Overall general | — |
| | performance | |
| Performance | System durability | — |
| over time | | |

TABLE IV
ABBREVIATIONS

| Abbreviation | Full form |
|---|---|
| BCI | Brain Computer Interface |
| ML | Machine Learning |
| EEG | Electroencephalography |
| EMG | Electromyography |
| MND | Motor Neuron Disease |
| ALS | Amyotrophic Lateral Sclerosis |
| SCI | Spinal Cord Injury |
| VC | Volume Conduction |
| SNR | Signal-to-Noise Ratio |
| fNIRS | Functional Near-Infrared Spectroscopy |
| PRISMA | Preferred Reporting Items for Systematic |
| | review and Meta-Analysis |
| CSV | Comma Separated Values |
| DOI | Digital Object Identifier |
| EOG | Electrooculogram |
| MI | Motor Imagery |
| ERD | Event-Related Desynchronization |
| ERS | Event-Related Synchronization |
| SSVEP | Steady-State Visual Evoked Potential |
| ERP | Event-Related Potential |
| MT | Mental Task |
| DC | Direct Current |
| CSP | Common Spatial Pattern |
| DFBCSP | Discriminative Filter Bank CSP |
| BP | Band Power |
| PSD | Power Spectral Density |
| BPF | Band Pass Filter |
| SVM | Support Vector Machine |
| LDA | Linear Discriminant Analysis |
| FLDA | Functional LDA |
| SWLDA | Step-Wise LDA |
| RBF | Radial Basis Function |
| HKF | Hybrid Kernel Function |
| CCA | Canonical Correlation Analysis |
| FBCCA | Filter Bank CCA |
| RMS | Root Mean Square |

TABLE V

| Spontaneous | MI & MT | [41], 2018 | 9 | (F, C, P, T) | 200 | — | Yes | — | — | None |
|---|---|---|---|---|---|---|---|---|---|---|
| | MI | [42], 2018 | 31 | (F, C, P) | 250 | — | Yes | — | Active | — |
| | MT | [43], 2017 | 14 | (F, P, T, O) | 128 | Wireless | — | Wet | — | Yes (custom) |
| | MI | [44], 2017 | 9 | (F, C, P, T) | 200 | — | Yes | — | — | None |
| | MI | [45], 2017 | 24 | | 250 | Wireless | — | Dry | — | — |
| | MI | [46], 2013 | 15 | (F, C, P, T) | 250 | — | Yes | — | — | — |
| | MI | [47], 2011 | 10- F, C | | 250 | — | Yes | — | — | — |
| SSVEP | | [48], 2021 | 8 | (O) | — | Wireless | Yes | Dry | — | — |
| | | [49], 2020 | 1 | (O) | 250 | — | Yes | Wet | — | — |
| | | [50], 2013 | 6 | (O, F) | 240 | — | — | — | — | — |
| P300 | | [51], 2021 | 12 | (F, C, P, O) | 256 | — | Yes | — | Active | — |
| | | [52], 2019 | 15- C, P, O. | | 200 | — | Yes | Wet | — | — |
| | | [53], 2013 | 12 | (F, C, P, O) | 256 | — | Yes | — | Passive | — |
| | | [54], 2010 | 15 | (F, C, P) | 250 | — | Yes | — | — | Yes (custom) |
| Hybrid | MI& SSVEP | [55], 2022 | 12 | (C, P, O) | 256 | — | Yes | — | — | — |
| | MI& P300 | [56], 2017 | 18 | (F, C, P, O) | 250 | — | Yes | — | Active | — |
| | MI& SSVEP | [57], 2014 | SSVEP: 4(P, O) | | 256 | — | Yes | — | — | — |
| | | | MI: 11(F, C) | | | | | | | |
| | MI& SSVEP | [58], 2014 | 15 | (F, C, P, O) | 256 | — | Yes | — | — | — |
| | MI & P300 | [59], 2012 | 15 | (F, C, P, O) | 250 | Yes | Yes | — | — | — |
| Multi-modal | MI &EOG | [33], 2019 | EEG: 9 (F, C) | | 250 | — | Yes | Wet | Passive | — |
| | P300 &EOG | [60], 2017 | 2 | (O, C) | 200 | Wired | Yes | Wet | — | Yes (mean |
| | | | | | | | | | | removal) |
| | MI, P300& | [61], 2014 | 15 | (F, C, P O) | 250 | — | Yes | Wet | — | — |
| | EOG | | | | | | | | | |
| | MI, P300 | [62], 2014 | 15 | (F, C, P, O) | 250 | — | Yes | — | — | — |
| | &speech | | | | | | | | | |

| | | | | | | | | |
|---|---|---|---|---|---|---|---|---|
| Spon. EEG | [63], 2012 | Headband | 512 | Wireless | Yes | — | Active | — |
| &blinking | | | | | | | | |

* F: frontal, C: central, P: parietal, O: occipital, T: temporal

EEG acquisition and preprocessing details for the articles in Table I shown above.

### Methods

### Dataset

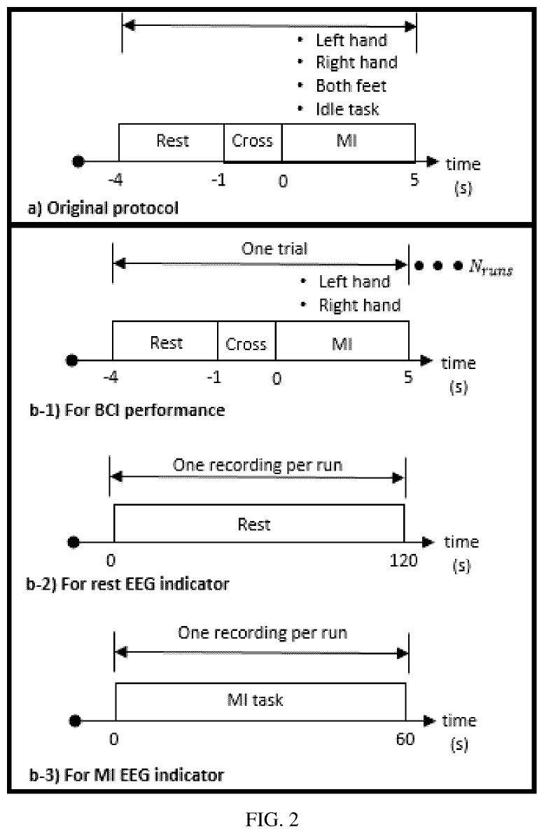

Published data was used for in the present disclosure [9]. Signals were collected using 64-channel Synamps2 system (Neuroscan, Inc.) with 500 Hz sampling frequency for 20 subjects (11 males, 23.2±1.47 years, all right-handed). On average, each subject conducted seven sessions, each consisting of six runs, and each run is composed of 20 trials of four mental tasks. The experiment protocol in the original work and the modified version of it used to produce this work are shown in FIG. 2.

Of the four mental tasks performed in the original dataset, only two were considered for this study: left-hand and right-hand movements. Also, sufficiently long periods of rest EEG signals were used. Since such periods were unavailable the original dataset, the 3-sec rest segments prior to each mental task in the original recordings were concatenated for each run. Then, random (120) 1-sec points were chosen from the concatenated recording to form (1) 2-min rest recording representative of that run. The same procedure was followed to obtain the MI indicators for both tasks, but the length taken was only 60s.

In the original dataset, either 41 or 26 EEG electrodes were used. Of those, 14 channels were considered.

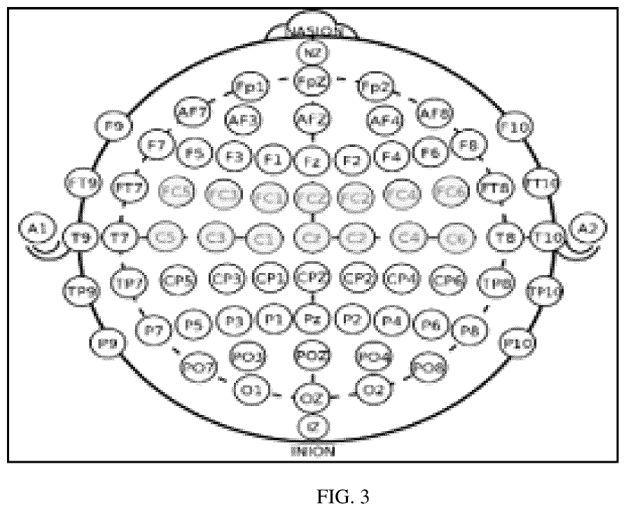

These were located in the central and fronto-central brain regions, over the motor cortex region responsible for initiating physical movement commands, as shown in FIG. 3. In addition to the EEG channels, two Electrooculography (EOG) electrodes were used to detect eye movement: horizontal EOG (hEOG) and vertical EOG (vEOG) electrodes. Both were used for removing eye blinking artifacts, as shown in the following subsections.

### MI Signal Processing

A classification scheme was built for the signals. This was done for two reasons including validating the dataset and test measuring its quality, and studying a possible correlation between the rest and MI-EEG signal characteristics and final performance.

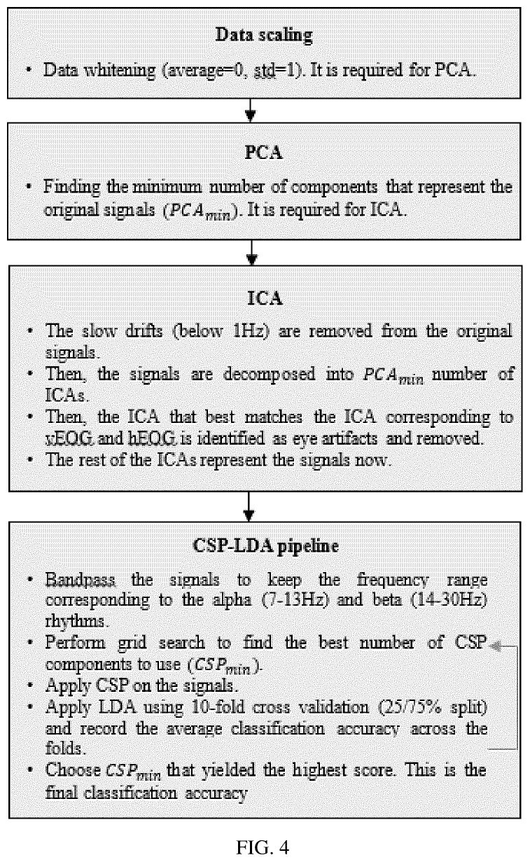

EEG signals are notorious for being susceptible to body artifacts and environmental noise. Thus, these undesired components were identified and removed from the EEG recordings before they were fed to the classifier. Also, the data needed to be reduced in size without losing in the desired neural information embedded in the signals. So, several signal processing steps were needed before the EEG signals were fed to the classifier. The steps are shown in FIG. 4.

The first three steps in the figure represent the preprocessing steps performed to remove eye artifacts from the recorded signals. First, the data were rescaled to have an average value of 0 and a standard deviation of 1. Then, Principal Component Analysis (PCA) was performed to find the minimum number of sources that contain 95% of the signals' variance. After that, Independent Component Analysis (ICA) was performed to remove the source representing the eye artifacts; EEG signals were decomposed into a PCA_min number of Independent Components (ICs) and the ICA that was most correlated with either of the EOG channels was eliminated from the set of ICAs. After this step, the remaining ICs resembled the original EEG signals after removing eye artifacts.

The last box in FIG. 4 resembles the feature extraction and feature classification steps. First, data was filtered to remove the unwanted range of frequencies outside of the alpha and beta rhythms. These were the brain waves representing the MI response of interest. Then, Common Spatial Patterns (CSP) was applied to further reduce data size. The number of CSP components was determined using grid search using 1-20 CSP components. The minimum number of components (CSP_min) that yielded the highest classification accuracy by the Linear Discriminant Analysis (LDA) classifier was chosen for that run. LDA classification was carried out using 10-fold cross-validation.

### EEG Indicators

Two types of indicators: rest and MI, are explained in this subsection. The rest indicators were obtained from the rest segments (see b-2 in FIG. 2), and the MI indicators were obtained from the MI recordings for each task (b-3 in FIG. 2).

The rest indicator was designed with [4] in mind. In [4], the SMR values were found. These values were the maximum PSD peak in the signal after subtracting the estimated noise floor for the different EEG channels. Each channel yielded a different SMR value. The Blankertz indicator was the average of the SMR values across several channels. The Blankertz indicator was found by iteratively solving a set of equations over 9 variables, which was a computationally expensive process, especially considering that this process was needed for all channels for each run the subject performed. Most importantly, the Blankertz indicator was limited by the stronger PSD between the two rhythms, alpha and beta. Thus, the authors of this study decided to use the PSD of the rest EEG signals for both the alpha and beta rhythms as rest indicators. Also, the indicators were found for each brain hemisphere individually, as it is known that different people may have different functionality levels in either of the hemispheres. Thereby, four rest EEG indicators were found and used herein, including:
PSD_(alpha, right, rest): average PSD in the alpha range averaged over the 6 EEG channels in the right hemisphere (FC2, FC4, FC6, C2, C4, and C6).
PSD_(alpha, left, rest): average PSD in the alpha range averaged over the 6 EEG channels in the left hemisphere (FC1, FC3, FC5, C1, C3, and C5).
PSD_(beta, right, rest): average PSD in the beta range averaged over the 6 EEG channels in the right hemisphere (FC2, FC4, FC6, C2, C4, and C6).
PSD_(beta, left, rest): average PSD in the beta range averaged over the 6 EEG channels in the left hemisphere (FC1, FC3, FC5, C1, C3, and C5).

For the MI indicators, the same type of indicators were found, yielding in 4 indicators for each of the two tasks. Since the difference in neural behavior was what was needed from the MI indicators; this was studied to find if it correlated with rest EEG or not. The difference between the indicators between the two tasks was found, resulting in the following MI indicators.
PSD_(alpha, right, MI): the difference between average PSD values in the alpha range in the right hemisphere among both MI tasks.
PSD_(alpha, left, MI): the difference between average PSD values in the alpha range in the left hemisphere among both MI tasks.
PSD_(beta, right, MI): the difference between average PSD values in the beta range in the right hemisphere among both MI tasks.
PSD_(beta, left, MI): the difference between average PSD values in the beta range in the left hemisphere among both MI tasks.

### BCI Performance

The accuracy values obtained by following the strategy outlined in FIG. 4 is shown in Table VI below. The results reported are averaged over the 42 runs performed by each subject (7 sessions×6 runs).

TABLE VI
THE CLASSIFICATION ACCURACIES ACHIEVED
IN THIS WORK FOR THE 20 SUBJECTS.

| | Avg. Acc. | Min. Acc. | Max. Acc. | Std. |
|---|---|---|---|---|
| Sub | [%] | [%] | [%] | [%] |
| S1 | 89.27 | 70.00 | 98.00 | 5.70 |
| S2 | 88.33 | 67.99 | 98.00 | 6.50 |
| S3 | 90.90 | 82.00 | 98.00 | 4.26 |
| S4 | 87.82 | 74.00 | 98.00 | 5.96 |
| S5 | 88.51 | 70.00 | 98.00 | 6.40 |
| S6 | 88.29 | 70.00 | 98.00 | 6.76 |
| S7 | 87.65 | 52.00 | 96.00 | 7.37 |
| S8 | 90.00 | 70.00 | 100 | 4.95 |
| S9 | 90.34 | 82.00 | 98.00 | 3.85 |
| S10 | 88.00 | 66.00 | 100 | 7.14 |
| S11 | 89.66 | 72.00 | 100 | 4.65 |
| S12 | 82.48 | 62.00 | 98.00 | 10.00 |
| S13 | 89.00 | 70.00 | 96.00 | 4.98 |
| S14 | 90.19 | 80.00 | 100 | 4.16 |
| S15 | 89.05 | 74.00 | 96.00 | 5.13 |
| S16 | 85.10 | 52.00 | 98.00 | 10.58 |
| S17 | 88.92 | 72.00 | 96.67 | 4.91 |
| S18 | 86.57 | 66.00 | 98.00 | 8.34 |
| S19 | 89.02 | 76.00 | 100 | 5.20 |
| S20 | 88.38 | 66.00 | 98.00 | 5.99 |
| Avg. | 88.37 | 69.70 | 98.13 | 6.14 |

MI classification problems, particularly considering the large number of trials performed by each subject. These results showed the adequacy of the signal processing technique used in this work and also show the legitimacy of the dataset obtained online [9]. Hence, it is valid to use this dataset for finding the correlations between rest and MI-EEG signals.

### Correlation Between Rest and MI-EEG

Now, the four indicators found from the rest recording were compared with the same four indicators found from the MI signals. The correlation was investigated by using the Pearson correlation coefficient (r). When r is above |0.5|, a strong correlation between the two sets of variables was said to be found. An r value that was less than |0.5| but above |0.3| indicated medium strength of correlation, and a value that was less than |0.3| but above |0.1| indicated weak correlation. The Pearson correlation coefficients for the 20 subjects are shown in Table II. The individual values were calculated based on all 42 runs performed by each subject.

All 20 subjects showed a medium or strong direct correlation for one of the indicators. 12 of the 20 subjects showed at least one strong correlation between the indicators. These results showed that higher PSD levels at resting-state resulted in a greater difference in the PSD levels between the two MI classes. For 3 subjects (S1, S6, and S11), correlations were only found in one of the rhythms. And 4 subjects (S3, S4, S11, and S15) only showed correlations for one of the brain hemispheres. These observations provided preliminary evidence for the value of investigating each brain hemisphere individually and on a rhythm basis.

TABLE VII
THE PEARSON CORRELATION COEFFICIENT BETWEEN REST
AND MI-EEG INDICATORS FOR THE 20 SUBJECTS. YELLOW
HIGHILIGHTING INDICATES STRONG CORRELATION AND
GREEN INDICATES MEDIUM CORRELATIONS.

| Sub | r_{alpha, left} | r_{alpha, right} | T_{beta, left} | T_{beta, right} |
|---|---|---|---|---|
| S1 | 0.18 | 0.26 | 0.33 | 0.42 |
| S2 | 0.55 | 0.44 | 0.28 | 0.44 |
| S3 | 0.24 | 0.37 | 0.21 | 0.42 |
| S4 | 0.27 | 0.34 | −0.04 | 0.77 |
| S5 | 0.25 | 0.37 | 0.32 | 0.20 |
| S6 | 0.22 | −0.02 | 0.77 | 0.87 |
| S7 | 0.70 | 0.74 | 0.70 | 0.74 |
| S8 | 0.51 | 0.68 | 0.35 | 0.15 |
| S9 | 0.70 | 0.44 | 0.75 | 0.27 |
| S10 | 0.49 | 0.53 | 0.55 | 0.42 |
| S11 | 0.18 | 0.24 | 0.44 | 0.43 |
| S12 | 0.46 | 0.42 | 0.41 | 0.21 |
| S13 | 0.87 | 0.76 | 0.70 | 0.61 |
| S14 | 0.16 | 0.44 | 0.02 | 0.10 |
| S15 | 0.10 | 0.38 | 0.27 | 0.50 |
| S16 | 0.31 | 0.38 | 0.42 | 0.52 |
| S17 | 0.11 | 0.17 | 0.37 | 0.26 |
| S18 | 0.74 | 0.17 | 0.63 | 0.45 |
| S19 | 0.35 | 0.28 | −0.10 | −0.20 |
| S20 | 0.58 | 0.46 | 0.57 | 0.47 |
| Avg. | 0.4 | 0.39 | 0.4 | 0.4 |
| Max | 0.87 | 0.76 | 0.77 | 0.87 |

### Correlation Between MI-EEG and Performance

The same MI indicators listed before were tested with the accuracies achieved to identify whether any correlation was observed. Logically, a greater difference between the MI classes should result in higher accuracies due to the higher level of distinction between the two. The r values generated by examining the relationship between the four MI indicators and performance are shown in Table VIIIA.

TABLE VIII
A: THE PEARSON CORRELATION COEFFICIENT
MI-EEG INDICATORS AND ACHIEVED ACCURACY
VALUES FOR THE 20 SUBJECTS. YELLOW HIGHLIGHTING
INDICATES STRONG CORRELATION AND GREEN
INDICATES MEDIUM CORRELATION.

| Sub | r_{alpha, right} | r_{alpha, left} | r_{beta, left} | T_{right, right} |
|---|---|---|---|---|
| S1 | −0.20 | −0.15 | −0.25 | −0.06 |
| S2 | −0.07 | −0.01 | −0.07 | −0.02 |
| S3 | 0.00 | 0.04 | 0.07 | 0.11 |
| S4 | −0.22 | −0.28 | 0.06 | −0.02 |
| S5 | −0.11 | 0.00 | −0.15 | −0.26 |
| S6 | −0.09 | −0.28 | −0.36 | −0.36 |
| S7 | 0.07 | 0.13 | 0.04 | 0.15 |
| S8 | 0.02 | 0.04 | −0.05 | −0.12 |
| S9 | −0.04 | 0.04 | 0.08 | 0.17 |
| S10 | −0.36 | −0.31 | −0.52 | −0.26 |
| S11 | −0.01 | −0.20 | 0.20 | 0.15 |
| S12 | −0.21 | −0.21 | −0.34 | −0.29 |
| S13 | −0.06 | −0.04 | 0.03 | 0.02 |
| S14 | 0.16 | 0.12 | −0.07 | 0.18 |
| S15 | −0.17 | −0.11 | 0.02 | −0.03 |
| S16 | −0.17 | −0.22 | −0.37 | −0.36 |
| S17 | −0.20 | −0.06 | 0.02 | −0.03 |
| S18 | −0.11 | −0.14 | −0.11 | −0.28 |
| S19 | −0.33 | −0.30 | 0.15 | 0.06 |
| S20 | 0.17 | 0.20 | 0.11 | 0.13 |

From Table VIIIA, only one strong correlation was found and only 5 subjects showed medium correlations. Furthermore, the correlations were negative indicating an inverse relationship; a greater difference in the MI indicators was associated with lower accuracies achieved, which is counterintuitive. Note that the indicators were based on the PSD values and classification was done using CSP features. This may explain this unexpected behavior. To investigate this point, the LDA classifier was reconstructed using the MI indicators instead of the CSP features. More correlations were observed in this case but the achieved accuracies were low (around the chance level of 50%) so these results are not reported here.

Strong and medium correlations were found between PSD indicators between resting-state and while performing MI tasks. The indicators were based on two brain waves: alpha and beta, and for both hemispheres of the brain. These correlations provide scientific evidence for the possibility of using rest EEG signals to obtain some information on the characteristics of the signals triggered by performing MI tasks. These correlations illuminate reasons behind the variance in the obtained results among the subjects and between the indicators extracted for different rhythms and brain regions. Also, the type of indicators may relate to PSD having limited significance in EEG signals. MI behavior (or rest behavior if the correlation is strong enough) along with the features used in the classification process may be related to correlation to minimize the classifier training requirements. This relates to strategies for matching a rest EEG indicator type and a classification strategy followed.

### Additional Studies

### From Technique Perspective (MI-EEG BCI).

Motor Imagery (MI) paradigms of Electroencephalography (EEG) have been used in developing assistive and rehabilitation devices, such as computer cursors (Kayikcioglu & Aydemir, 2010), artificial limbs (Horki et al., 2011), and wheelchairs (Tsui et al., 2011; H. Wang & Bezerianos, 2017; Yu et al., 2018). A direct link between a human brain and external tools and devices offers opportunities to people with special needs, as discussed herein. Former attempts have not been successful, however, in translating lab results to real-life scenarios (Aricò et al., 2018; Douibi et al., 2021).

Failures of previous attempts relate to the current framework requiring a pre-use data collection phase before the final application is completed (Padfield et al., 2019) and the continuously-changing characteristics of EEG signals, known as nonstationarity behavior. For example, someone's age induces neural changes that in turn affect EEG (Al Zoubi et al., 2018; Schott, 2012). Several other dynamic factors are contributing to neural behavior changes including emotional status, which by itself is a field of research (Alarcao & Fonseca, 2019; Ko et al., 2009).

The dynamic nature described above necessitates training data not just once but for every nonstationarity contributing factor, such as changes in mood, focus, and every possible mental state, which is practically impossible to achieve. Such issue is important in assistive technologies. An end-user, who is a person with some form of disability, has difficulties performing long, tiring data collection sessions, during which a large number of MI tasks should be performed and recorded before every use of the model. This impracticality has led to a shift in interest towards reactive EEG paradigms over MI (Värbu et al., 2022), and casted doubt on the prospect of utilizing MI-EEG for control applications.

Nonstationarities may be significantly challenging to MI-EEG. Addressing this issue has the potential to dramatically increase the adoption rates of MI-EEG in the field of Brain-Computer Interaction (BCI), given its versatile applications.

The present disclosure provides a novel approach to the MI paradigm, as further described herein. The present disclosure provides a framework with for making EEG-MI predictable by utilizing Rest signals despite EEG-MI's non-stationary challenges. The disclosure provides a design and framework that is based on more retrievable EEG signals. This provides a framework that is more applicable in real applications for many rehabilitation and assistive technology developments.

From application perspective (BCI-driven wheelchairs): BCI-driven wheelchairs bring hope to hundreds of thousands of people around the globe, as previously described. In this use, different MI tasks (mentally performed limb movements) are linked with different navigational commands of the chair. For example, a right-hand MI is linked to a right-hand turn by the chair, and a left-hand MI is linked to a left-hand turn, etc. This use provides a solution for individuals with both upper and lower body disabilities for whom it is not possible for them to control wheelchairs via joysticks.

BCI wheelchairs hold the potential to benefit diverse populations, including those with Multiple Sclerosis (MS), Spinal Cord Injuries (SCI), such as quadriplegia, Motor Neuron Diseases (MND), such as Amyotrophic lateral sclerosis (ALS), and more. While recognizing the applicability of EEG technology, wheelchair uses are limited given the challenges associated with working with EEG. The present disclosure provides a solution to overcome such challenges. In some models, some form of human input is needed, and the type of this input directly impacts the suitability of specific models for accommodating diverse populations with varying needs. The available techniques for hands-free wheelchairs are outlined in Table VIIIB (Leaman & La, 2017; Phinyomark et al., 2011; X. Zhang et al., 2021).

TABLE VIII
B. AVAILABLE HANDS-FREE WHEELCHAIR MODELS.

| | | Physical | | |
|---|---|---|---|---|
| | | activity | | Suitable |
| Group | Human input | needed | Requirements | conditions |
| 1 | EOG (Electrooculography), | Yes | Differs based | Incomplete SCI, less- |
| | Eye movement, Vision-based | | on type | severe MS, etc. |
| | (Head/hand gestures), | | | |
| | Tongue actuation, Sip-and- | | | |
| | puff, Voice recognition | | | |
| 2 | EMG (Electromyography) | No | Unimpaired | Based on the location |
| | | | muscle nerves | of electrodes |
| 3 | EEG | No | Unimpaired brain | Complete SCI, severe |
| | | | functionality | MS, ALS, etc. |

The first group in the table, which involves activity in the head region, is not effective for severe conditions. In such cases, only EMG and EEG are viable options. Among these, EEG holds an advantage over EMG, as the latter relies on functionality from the muscle nerves, whereas EEG only requires a functioning brain. The brain is the last functioning organ in human beings and the source of humanization. If the brain is compromised, efforts to assist in mobility become futile. Therefore, the PI asserts that EEG is the preferred modality for several conditions where targeted muscle nerves are compromised, such as the case of ALS (Mayo Clinic, 2023). This shows that EEG remains a practical choice for the ALS population and is viable for other conditions until severe brain damage occurs.

The present disclosure relates to wheelchair implementations of findings described herein. This allows the disclosure contained herein to directly address end-user populations in real-world uses, which is not common in the MI-BCI field. The distance from the user space of previous works neglects the potential impact of various conditions on model design. For instance, ensuring that EEG signals from users with neural conditions can perform MI tasks similarly to those without the conditions is uncertain and raises questions about the validity of modifying existing models to work with them. The scarcity of publicly available data for such populations adds hurdles to initiating this research per previous works. The present disclosure addresses these issues.

The present disclosure provides real-world uses that engage end users. This is achieved through computational work, identifying unique signal characteristics, assessing the feasibility of modifying existing models based on their signals, and understanding user needs in such an application. A wheelchair application is intended as a non-limiting example and is not exclusive. Methods, processes, and systems disclosed herein are applicable to any BCI application and are not restricted to the ALS population, as existing datasets and a large number of subjects without neural conditions are disclosed herein.

As a non-limiting example, the present disclosure relates to subjects with ALS. Among the various conditions that BCI wheelchairs can serve, a singular condition allows the present disclosure to illustrate the importance of involving a subject from the modeling stage. ALS provides a basis for a high likelihood of influencing MI-EEG, which enables further expansion of the present disclosure to other groups. MNDs affect motor neurons responsible for initiating MI signals. Within the category of MNDs, to avoid confounding effects from multiple conditions, the focus was narrowed down to the most prevalent MND, which is ALS.

Even though there are no exact numbers for people with combined upper and lower body disabilities available, the prevalence of ALS, the most common disease causing both upper and lower motor neuron degeneration, was recently estimated at 9.1 per 100,000 U.S. population (Mehta et al., 2023). It was also estimated to be 4.45 per 100,000 population worldwide (L. Xu et al., 2020). And it is expected to keep growing in the coming few decades (Arthur et al., 2016). ALS is one type of MNDs that include seven medical conditions (WebMD Editorial Contributors, 2020). There are approximately 268,673 prevalent cases of MNDs worldwide (Park et al., 2022). Other conditions such as SCI and MS are also prevalent. SCI are estimated at 17,000 new cases of SCI each year, and roughly 282,000 persons are estimated to be living with SCI in the U.S. (“Spinal Cord Injury (SCI) 2016 Facts and Figures at a Glance,” 2016). Also, the incidence of MS is around 300-450 per 100,000 individuals (Wallin et al., 2019).

### Rigor of Additional Studies

The present disclosure relates to a number of aims, as described herein. Provided below are details relating to previous works that relate to a number of aims. Novel methods, processes, systems, and the like relating to aims of the present disclosure are described later in the present disclosure.

### Example 1: Quantify the Relationship Between Rest and while Performing MI Tasks

The logic behind the ability of Rest to predict MI characteristics is based on the following observations. The characteristics of MI signals are influenced by two main elements. The first component is biological, stemming from the inherent structure of the human body. It involves biomarkers associated with various mental states and the activity related to maintaining muscle tone and responsiveness. Essentially, this reflects a specific momentary state of the individual. The second element is the imaginative capability of the individual-how vivid, robust, and consistent their imagination is over a given time frame. Importantly, this imaginative capacity correlates to the first component, which is the individual's current mental state. The first element is responsible in part for the characteristics of MI signals. This is what is being captured by the Rest state, which can also be thought of as a neural ID of the subject at a given point in time. The variability in imagination between subjects can be mitigated by guiding the subject during the data collection. This guidance involves providing real-time video of the task being performed, ensuring that participants consistently imagine the task throughout the entire trial period. Thus, the imagination capability is not as vital as the first biological component that encompasses many contributing factors. This biological component is reflected in the Rest state.

The value of Rest in determining MI characteristics is based on several publications concerned with BCI illiteracy. In those articles, classifier performance was predicted from Rest-EEG recordings to identify individuals with low potential of generating distinctive MI signals for different MI tasks. Though these efforts demonstrate the value of resting-state signals, they do not link Rest to MI signal characteristics because they do not relate to minimizing or eliminating training requirements. The connection such publications established between Rest and performance is dependent on the MI-EEG signals. Hence, these publications directly support the influence of Rest on the generated MI signals. Examples of such publications are discussed below.

The Blankertz group from the Technical University of Berlin (Blankertz et al., 2010) has shown a Pearson correlation coefficient (r=0.53) between a Rest-EEG indicator, that is based on Power Spectral Density (PSD), and classification accuracy. The strength of two Laplacian channels over the motor cortex were identified to exhibit a direct linear relationship with the accuracies achieved when utilizing band power features. An increase in the average Rest-EEG power corresponds to an increase in the MI-EEG strength across different classes. The correlation value reported is based on a gross examination of 80 subjects, where each subject is treated as a single point. The subjects included both males and females and for three MI classes (right hand, left hand, and feet), which adds credibility to the results, as achieving higher correlations becomes more challenging with a larger number of classes and when dealing with gender as a biological variable.

The work of Ahn (Ahn et al., 2013) has shown a Pearson correlation coefficient (r=0.59) between a Rest-EEG indicator and classification accuracy using Common Spatial Patterns (CSPs) as features. As is the case with (Blankertz et al., 2010), the correlation value reported is based on the whole set of subjects considered (61 subjects, both males and females), but for two MI tasks (both hands). The indicator is also strength-based using the band powers of Theta and Alpha (integration of PSD in the band's specific frequency range).

The work of (R. Zhang, Xu, et al., 2015) has shown a Pearson correlation coefficient (r=0.65) between a Rest-EEG indicator and classification accuracy for 26 subjects (males and females), using CSPs as features. In their work, they utilized the spectral entropy generated based on the PSD of the Delta, Theta, and Alpha rhythms of the resting-state EEG recording. As is the case with (Blankertz et al., 2010) and (Ahn et al., 2013), the correlation is reported based on all subjects' data. Also, when the Blankertz and Ahn indicators were tested in (R. Zhang, Xu, et al., 2015), they were shown to have r=0.29 and r=0.51 correlation coefficients, respectively. The first value corresponding to the Blankertz indicator is drastically lower than the original work. This may result from the type of features used (CSP rather than band powers). When using band power features, which are strength-based features akin to the Rest indicator, it is easier to observe the correlation between Rest-EEG and final performance. However, if more advanced features are utilized, such as CSPs or NN-based features, the process of transferring this knowledge from the Rest phase into a meaningful and well-manifested form within the extracted MI features becomes a more challenging task. However, this investigation of the first two indicators still shows the value of Rest in determining BCI performance.

Other work on BCI illiteracy include (Bamdadian et al., 2014; Grosse-Wentrup & Schölkopf, 2012; Jeunet et al., 2015; Lee et al., 2020; R. Zhang, Xu, et al., 2015; R. Zhang, Yao, et al., 2015). In addition to BCI illiteracy studies, (Naser & Bhattacharya, 2023) displayed similar patterns. In this work, four features were extracted for Rest and ΔMI representing the PSD in both the Alpha and Beta rhythms for both hemispheres. Then, the Pearson correlations were found between the features from both states. All 20 subjects show at least one medium (|0.5|≥r≥|0.3|) or strong (r≥|0.5|) direct correlation, and 12 of which show a minimum of one strong correlation. This means that higher PSD levels at Rest result in a greater difference in the PSD levels between the two MI classes. These results demonstrate the potential value of resting-state signals in predicting the characteristics of MI signals. While the strength-based features introduced above indeed demonstrate a robust correlation between Rest and MI, they may not be the optimal features for correlating the states together. Therefore, thisexampleis not only concerned with finding the correlations but also quantifying those and explaining how to go from one state to the other.

The work of (Zanini et al., 2018) is the only known work to minimize training requirements using Rest signals. It was done using a Riemannian alignment approach. The authors focused on feature-level Transfer Learning (TL) by centering the covariance matrices of MI classes related to different subjects and captured in different sessions on the Riemannian manifold, using Rest as a reference point. Testing was conducted on both a session and subject basis. In the former case, the results demonstrated an accuracy increase of up to 10% over the baseline (without transformation using Rest), while lower results were obtained for the subject-to-subject paradigm. Nonetheless, this still proves the value of Rest in training minimization.

The publications referred to in this subsection will not be replicated in the present disclosure as they serve a different purpose. They are included here to substantiate the argument regarding the validity of investigating Rest as correlated with MI characteristics.

### Example 2: Incorporate Rest Knowledge into an MI-EEG Modeling Framework to Minimize End-User Training Requirements

The value of Rest is discussed in the previous aim. This example relates to using Rest to minimize training requirements. The techniques proposed in the present disclosure are based on four components, and below is a review of previous research utilizing each of the components.

### Synthetic EEG Data Generation.

Synthetic EEG data has been explored in studies across different EEG paradigms. In the work of (Carrle et al., 2023), which analyzed 27 EEG synthetic data generation studies, the augmentation of synthetic and real data demonstrated improvements in accuracies ranging from 1% to as high as 40%. The authors further utilized Generative Adversarial Networks (GAN) to generate data for two datasets, resulting in a 10% improvement for one dataset, while the other showed minimal enhancement. Thus, this work has shown that synthetic data brings improvement to MI decoding models, even if marginal.

### Modifying Classification Pipelines for TL.

One classification pipeline is the CSP-LDA pipeline, where LDA is Linear Discriminant Analysis used as a classifier. Minimizing the need for training data on the CSP feature level, and on the LDA classification level has been done in publications in the literature. Such examples include (Hyohyeong Kang et al., 2009) where a composite CSP was created by weighting the covariance matrices of the different subjects, and found that this technique provides higher performance values when the number of labeled testing data is limited. Another work is (Samek et al., 2013) where the authors developed an invariant version of CSP, resilient to nonstationarities, by adding a penalty term to the CSP objective function. This penalty strategy was applied to penalize a Principal Component (PC) version of the differences between training and unlabeled testing data, resulting in most subjects scoring around 5% higher than baseline. A similar approach was taken in (Benjamin Blankertz et al., 2007) where a different method of quantifying nonstationarity was used and found to yield slight improvement in mitigating the effect of nonstationarities.

In (Devlaminck et al., 2011), the authors built a CSP model based on a large pool of data. Then, the CSP objective function was regularized by adding two terms that control “how global or local” the filter is based on labeled testing data points, resulting in around 70% accuracies and above. In (Lotte & Guan, 2010), the authors regularized both CSP and the classifier LDA towards certain data points. The main goal of that is to reduce the calibration time for a new subject by using data from a certain group of other subjects coupled with a small subset of the new subject's data. When the number of trials from the subject is low, the proposed algorithms yielded around 2% increase in accuracy compared to the baseline (subject-specific model). And in (Lotte & Cuntai Guan, 2011), the authors proposed four versions of regularized CSP algorithms and reviewed other regularization techniques in the literature. The work was concluded by stating that regularizing CSP can yield improvement, with accuracy increases reaching up to 10%. Similar techniques of regularizing objective functions can be extended to other pipelines, such as those composed of neural networks.

### Traditional TL in NNs Using Layer Freezing.

This is the traditional definition of TL in the machine-learning world. Such examples include (Mattioli et al., 2021) where the authors constructed an end-to-end convolutional neural network (CNN) where the input to the network is unprocessed EEG data. They performed TL by freezing the first 3 layers from training and completed the pipeline with the new subject data. The pipeline produced results around 50% (chance level is 20% for the 5 tasks considered). A similar technique was used in (F. Xu et al., 2021) using CNNs where the output layer was modified for the new testing data point and achieved around 65% accuracy values.

### Raw Data Alignment.

The research to support this point is extracted from the TL space performed on raw data, either in the Euclidean or Riemannian space. For the first type, there is (Chen et al., 2022) where the authors used the transport theory to jointly adapt the marginal and conditional distributions across domains in latent space by minimizing the Wasserstein distance between them, using only unlabeled testing data, achieving accuracies around 70%. In (X. Wang et al., 2022), the author found an alignment matrix to minimize the distance between the mean of features between the source and unlabeled target data points. The best pipeline achieved 67-87% accuracies. The literature includes alignment techniques, but the Riemannian space is not of interest to the present disclosure, as it limits the classification steps afterward.

### Example 3: Examine ALS Effects on EEG Signals and Adapt Models from Non-ALS Individuals for Effective Use in an ALS Group

The motivation behind this example is the lack of studies examining the potential difference in the characteristics of EEG signals between people with and without ALS, or any type of MNDs. Since MNDs affect the neurons responsible for MI tasks (National Institute of Neurological Disorders and Stroke, 2023), substantial differences are expected, including signal strength, duration of time users can perform MI tasks before it becomes unbearable, and others. That said, no peer-reviewed articles have shown a comparative study on this particular aspect of EEG. This is notable, knowing that most work in the literature adopts the use of data obtained from people with no physical disabilities to develop, test, and assess the performance of technology intended for people with disabilities (Naser & Bhattacharya, 2023b).

Only a few researchers have investigated MI-powered applications using people diagnosed with MNDs. Two published articles that examined EEG engineering applications using individuals with MNDs are (Cruz et al., 2021; Puanhvuan et al., 2017). Such publications use the reactive P300 paradigm for navigating a wheelchair, not MI. Very little information can be inferred from the publications because of the difference in the paradigm considered. Extending observations from P300 to MI is a questionable tactic as they have different underlying physiological dynamics. In addition, they do not examine the difference in neurological behavior between the two groups. So, even if a change in overall performance is revealed in these performance-oriented articles, it is not possible to determine whether or not it is the product of neurological variations between the two groups. This is due to the various external factors that affect the final performance recorded during the navigation trials of the chair.

### Significance of the Expected Research Contribution

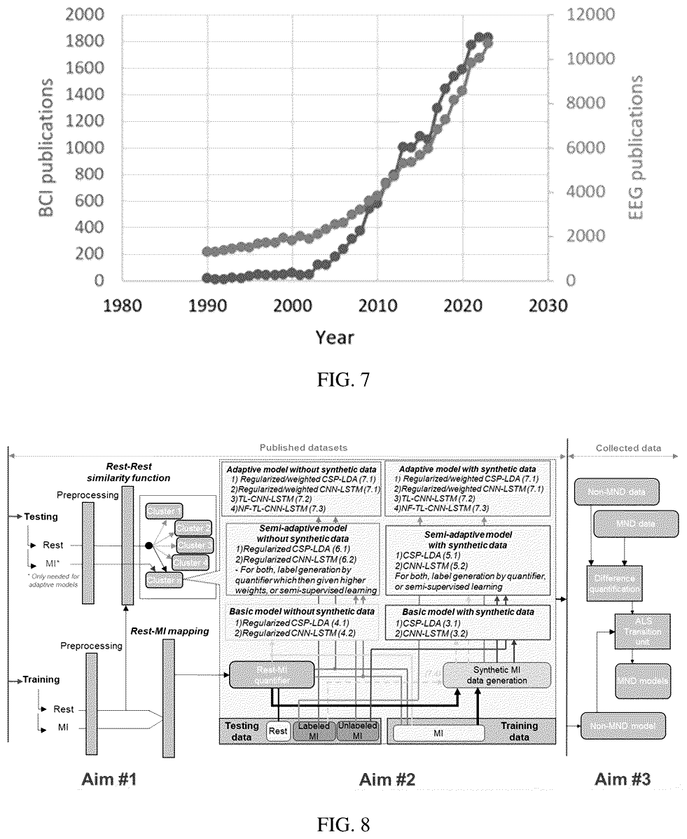

The BCI field has seen growth in the last two decades, as shown in FIG. 7. However, its fundamental system design poses a significant obstacle. Achieving vertical advancement is improbable without tackling EEG's major issue-signal nonstationarity, which the present disclosure addresses. This necessitates an adaptive, easy-to-obtain paradigm according to the present disclosure. Otherwise, BCI remains a theoretical concept in literature, impractical for real-time applications. This project specifically addresses this challenge and is expected to deliver the following novel contributions, as further described herein:

### Direct Contributions.

Showcase the effectiveness of Rest in alleviating nonstationarities through an 18-month-long investigation to quantify this relationship.

Develop several user training minimization models, diverging from those found in the literature, utilizing Rest as a neural ID to switch models seamlessly between different usage times and subjects.

Illustrate the feasibility of adapting models initially designed for individuals without neural conditions to function effectively within the targeted ALS population.

### Indirect Contributions.

Contribute to the community by providing an open-access EEG dataset for individuals with ALS. Given the resources and arrangements required to obtain it, this dataset is invaluable for the research community. In addition to that, all codes developed will be publicly available.

Offer additional evidence supporting the significance of creating user-involved technologies that originate from their distinct conditions and unique needs.

Opening a new era of MI-driven assistive technologies that go beyond simple proof-of-concept studies and into a more feasible approach for adoption.

### DETAILED DESCRIPTION

The present disclosure relates to the use of MI instead of reactive paradigms like P300 and SSVEP. The latter two paradigms are non-intuitive, necessitate additional equipment, and entail low response times, potentially discouraging users. Since MI is more intuitive, the present disclosure relates to utilizing the paradigm (such as MI commands linked to directional commands) as an EEG paradigm for controlling a wheelchair by members of the physically disabled community. Members of such community prefer MI based on a survey conducted according to the present disclosure at Kennesaw State University (KSU) under IRB #FY22-572.

The present disclosure relates to a software component developing MI-EEG decoding models. As a non-limiting example, the present disclosure displays a user performing a mental task unknown to a model, which analyzes EEG signals to identify a performed task. An identified task can subsequently be linked to various wheelchair functions (such as a hardware component), such as left or right direction control, speed adjustment, and more. The present disclosure relates to integrating both MI-EEG and supplementary modalities, including EMG and functional Near-Infrared Spectroscopy (fNIRS), to achieve a fully functional wheelchair system.

The present disclosure relates to EEG-powered applications and establishes a novel ground for utilizing MI-EEG signals in developing medical technologies. The present disclosure relates to developing pre-trained MI-driven wheelchairs that are practical for physically disabled individuals incapable of operating powered wheelchairs via joysticks. In such applications, rest-EEG signals are immediately recorded before model use, serving as a transition mechanism for deploying pre-trained models to new users, and bridging variations in signal behavior linked to the user's mental state, providing a pathway for more practical implementation of MI-EEG in real-life settings. Following are example, specific aims of the present disclosure, as discussed herein:

Aim 1. Quantify the relationship between Rest and while performing MI tasks. A Rest-MI quantifier is developed to quantify the relationship between the characteristics of both signal types. This may be accomplished using, as an example, three existing datasets and using various techniques applied to the raw data and handcrafted features based on time, frequency, and channel location.

Aim 2. Incorporate Rest knowledge into an MI-EEG modeling framework to minimize end-user training requirements. Several processing techniques, driven by synthetic data generation and Rest signals, are developed. Three levels of adaptation are implemented, ensuring continuous model improvement with incoming MI data. An adequate (minimum accuracy=75%) MI decoding model can be attained by solely utilizing the user's Rest data and pre-trained MI models from other subjects.

Aim 3. Examine Amyotrophic Lateral Sclerosis (ALS) effects on EEG signals and adapt models from non-ALS individuals for effective use in an ALS group. Participants diagnosed with early-stage limb-onset ALS (lo-ALS) and individuals without neural diseases will be recruited for data collection. A comparative analysis of their EEG signals aims to identify significant differences between the two groups. Signal strength in the ALS group is significantly lower than the other group, necessitating the redesign of non-ALS models to ensure their applicability to those with ALS.

The present disclosure develops an innovative MI framework that is resilient to nonstationarities. The present disclosure assesses the importance of involving end-users of EEG assistive technologies in the design process from the project's inception rather than incorporating their input as an afterthought. The present disclosure provides benefits to disabled communities by providing novel MI-EEG methods, processes, and systems. The present disclosure relates to, novel considerations for electric wheelchair BCI applications.

### Innovation

Traditional methods of using the MI paradigm of EEG in control applications consists of conducting an MI data collection session for every potential user to have a working machine learning model. Though this approach is valid, it has limited its deployment outside of research environments (Aricò et al., 2018; Jeunet et al., 2016; McFarland & Vaughan, 2016). For disability applications, this places an enormous burden on physically disabled users. This has discouraged many researchers from pursuing MI-EEG for controlling applications.

The present disclosure provides a novel solution by not conducting an MI data collection session for every potential user. The present disclosure provides a method of utilizing MI-EEG signals obtained elsewhere with a short segment of Rest-EEG obtained from the user in real-time to alleviate the need for MI training sessions. The move from MI training to utilizing Rest is significant, given that this transition is not a straightforward process. Rest represents a singular signal type attempting to substitute multiple signal types triggered by actions that are not present during Rest.

The present disclosure provides methods applicable to any application utilizing the MI paradigm of EEG, such as prosthetics (AL-Quraishi et al., 2018) and wheelchairs. This will shift the focus of traditional methods and translate EEG research into novel, unexplored areas to provide more practical assistive technology applications.

### Approach

FIG. 8 displays a framework of methods according to the present disclosure. The framework operates as follows: First, combined data points from all datasets are divided into training and testing sets. The training set is utilized to quantify the Rest-MI relationship, resulting in a set of rules representing what is called a quantifier. The Rest data from the training set is also employed to generate Rest-Rest metrics, forming clusters of data sharing similar Rest characteristics. This metric also facilitates matching a new testing data point to a cluster. Within these clusters, six types of methodologies are proposed, represented by distinct blocks in the figure and categorized as basic, semi-adaptive, and adaptive. Basic refers to models without any use of MI testing data, semi-adaptive refers to models utilizing unlabeled MI testing data, and adaptive refers to models utilizing labeled MI testing data. Each method employs different combinations of available data and techniques, as indicated by the varied colors of the arrows. Once the models are constructed, data is collected from both ALS and non-ALS groups. The latter is gathered to directly validate the models, while the ALS group is compared with the non-ALS group to quantify any differences and subsequently refine the constructed models. Modules, steps, and functions displayed in FIG. 8 are intended to provide examples of methods according to the present disclosure. Such modules, steps, and functions are non-limiting and may be augmented (e.g., add more steps), reduced (e.g., remove steps), or re-organized (e.g., re-order steps) according to the present disclosure.

Rest alone may not provide sufficient information to effectively control a system. Rest represents a type of signal recorded in the absence of intentional mental tasks performed by the individual. Control involves distinguishing between multiple EEG signals to associate each with a distinct command for the technological component (e.g., a wheelchair). These signals originate from performing multiple MI tasks including but not limited to right-hand and left-hand motor imagery tasks. In such examples, Rest serves as a guiding metric, representing a real-time mental state identifier, and is used with pre-collected MI data from other subjects to create a working model. In other words, Rest is not replacing MI. Rest is alleviating the need for MI data from the testing data point. It facilitates accurate prediction of real-time MI tasks through the utilization of pre-obtained MI signals.

Aim #1: Quantify the Relationship Between Rest and while Performing MI Tasks.

Using resting-state signals to substitute MI signals begins with establishing the relationships between these two states. This allows guiding the generation of MI signals for the testing subject in the following steps. The techniques employed for this purpose include trying to compare two types of data including Rest and MI data. The same techniques will be applied to compare other types of EEG data: Rest signals for two data points. Comparing Rest signals of two different data points is done for at least two reasons including but not limited to: 1) clustering the available data points and 2) matching a new data point representing a testing data point with cluster data. Cluster data includes information from either different subjects or from the same testing subject but recorded at an older time.

Aim 1 accomplishes at least two objectives including but not limited to: a) Rest-MI relationship quantification and b) Rest-Rest similarity metrics generation. example 1 determines the nature of connection measures to use throughout the methods described herein. No issues in linking the two Rest states together are expected. Potential inconsistencies in Rest and MI correlations among subjects, such as individuals showing different correlation measures between Rest and MI states, is overcome according to the present disclosure by the following: 1) conducting the modeling part in clusters based on stratified correlation patterns between the states and 2) providing access to testing MI data and utilizing Rest as an auxiliary guiding input in two of the three models developed in the next example (i.e., example 2) described below. The present disclosure provides an understanding of how to quantify the relationship between someone's Rest and MI, as well as how to link two Rest data points together, through example 1.

### Research Design.

### Data Used.

As an example, three published datasets of individuals without a history of neural conditions were used. The decision to initiate the project with published data prevented any potential delays that may have arisen while waiting for the completion of the data collection process. The other reason was to have benchmarking datasets for comparison with other methods in the literature. The choice to proceed with three different datasets was to ensure the robustness of the developed models to a variety of experimental conditions and any models' biases toward certain hidden data distributions and assumptions, enabling cross-validation using a large number of data points.

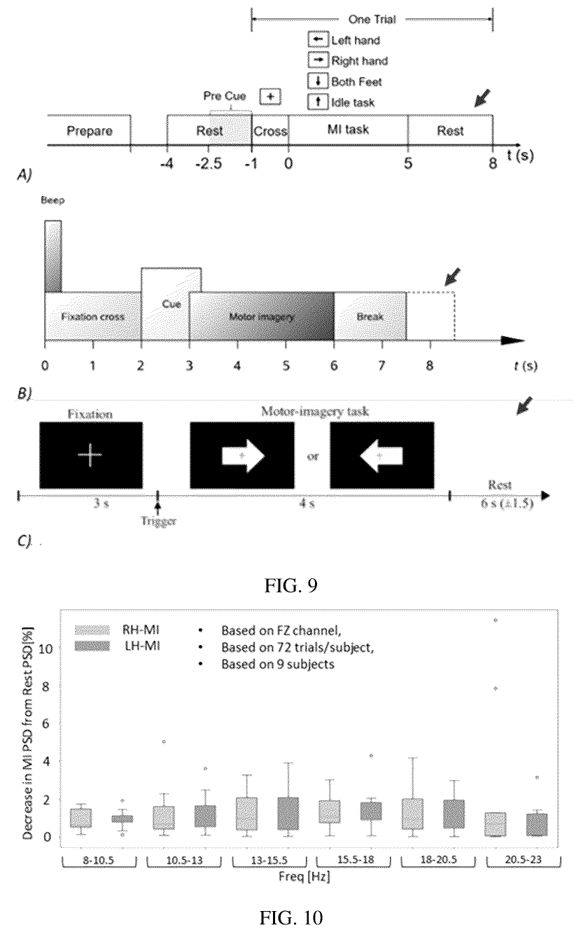

The datasets included (Zhou, Qing, 2020), which had already been used in generating the preliminary results introduced later on, the second set in (Deep BCI, 2017), a well-known and widely used dataset, and the most common MI dataset available: BCI competition IV 2a (BCI Competition IV, 2004), totaling in 73 subjects from all sets. As this example was the first to examine Rest in MI training context, the scope was limited to two types of MI tasks including Left Hand (LH) and Right Hand (RH) tasks. The chosen datasets had these two types of tasks with a total of around 1084 trials of each type per subject, recorded at different times and using high-quality acquisition devices. FIG. 9 shows the experimental paradigms for the three datasets used in example #1 including: A) (Zhou, Qing, 2020), B) (Deep BCI, 2017), and C) (BCI Competition IV, 2004). The arrows show the periods to use for Rest-EEG. None of the data contained in those sets intentionally recorded Rest segments, but they all included Rest segments recorded as a break between the trials without any visual stimulus coming from fixation period. So, these were used for Rest calculations.

Alternatively, other published datasets are capable of being used. Other datasets are available for replacement, but some examples of other datasets include Rest periods that are either shorter or presented with the presence of a visual cue. As an additional alternative example, the third dataset (BCI Competition IV, 2004) includes distributions that are known and can be used alone for example 1. This dataset is the largest, constituting 39% of the subjects' data. In such a scenario, the statistical analysis will be modified accordingly.

### Preprocessing.

All data processing and computational work was done in Python, chosen due to its richness with pre-built toolboxes and wide use. Then, EEG preprocessing was performed (including filtration, EOG artifact removal: PCA, ICA, regression, epoch rejection, and the like). The (Deep BCI, 2017) is an exception as the original work did not use EOG channels, so the frontal EEG channels were used for that purpose.

Stratified analysis was performed using composite stratification layers defined by gender (males and females) and age groups. A 10-year age stratification was implemented, resulting in layers such as 18-28 years old males, 18-28 years old females, and so forth. The utilization of this stratification approach addressed the impact of gender and age as contributing factors on EEG signal characteristics. This stratification was consistently applied throughout the entire example.

### Rest-MI Quantifier.

This unit comprises a set of empirically defined rules outlining the relationship between the two states. It includes a list of guidelines to predict the characteristics of MI from Rest. Examples of such guidelines might be: ‘The distinction between Rest and RH-MI is that MI signals exhibit x₁% (CI: xₗ_{₁}%-xₕ_{₁}%) higher power in a specific frequency range in the left hemisphere channels and x₂% (CI: xₗ_{₂}%-xₕ_{₂}%) lesser power in the right hemisphere,’ or ‘During LH-MI, the right hemisphere channels exhibit x₃% higher coherence than the left hemisphere and x₄% (CI: xₗ_{₃}%-xₕ_{₃}%) higher than Rest.’

Essentially, these rules were derived to provide insights into the corresponding MI characteristics based on the Rest state. To do so, both handcrafted features and more fundamental, statistical techniques performed on the time-series raw data were performed, to capture as many patterns as possible.

### Features.

The present disclosure relates to a variety of features of the three example data sets. 1) Basic strength-based features: Power Spectral Density (PSD), 2) Time-frequency features via Morlet wavelet decomposition, 3) Tempospatial features such as covariance matrices, 4) NN parameters, and 5) functional connectivity metrics, such as Coherence, Phase Locking Value, etc.

### Raw Data.

As an example, several methods were applied to the three data sets including: 1) Dynamic-Time Warping (DTW) algorithm, 2) Kullback-Leibler (K-L) divergence, 3) Kolmogorov-Smirnov (K-S) test, 4) Distance similarities (Euclidean, Manhattan, cosine), and 5) Quantile comparison. Since it imposing any statistical distribution on EEG time series may be difficult, all methods used were non-parametric. Furthermore, they operated on different underlying principles allowing the methods to be capable of complementing each other, along with observations based on the features.

In addition, these measurements were applied to the Rest-Rest case. In addition to the methods above, other similarity methods were used for the Rest-Rest case, these were the Mann-Whitney Test and the Wilcoxon Rank Sum Test. While these two tests treat observations as independent samples and neglect their temporal structures, they were still used to compare the descriptive summaries of EEG time series. Also, rather than relying on a single method, achieving a consensus among multiple methods was considered for obtaining consistently homogeneous groups of data points.

### Preliminary Data.

Aim 1 involves the Rest signals' ability to deduce MI characteristics. This translates into the principle that ‘similar Rest leads to similar MI,’ which forms the foundation of the clustering process and provides the potential to substitute the requirement of testing MI data with testing Rest data in the subsequent aims described below.

In support of this claim, FIG. 10 illustrates the results derived from the work performed on the (BCI Competition IV, 2004) dataset. FIG. 10 shows preliminary data on quantifying MI characteristics from Rest. PSD values for both Rest and MI states were obtained in 2.5 Hz increments. The x-axis delineates the frequency windows, while the y-axis represents the PSD difference between Rest and MI tasks. Notably, PSD values during MI were lower than those at rest due to Event-Related Desynchronization (ERD), resulting in a decrease in PSD while performing the MI task.

Each whisker plot in FIG. 10 depicts the values for the 9 subjects within the dataset. The figure indicates that, for most subjects, MI PSD strength falls within a range of approximately 0.01% to 2% less than the PSD of their Rest signals. More simply, having the PSD values of individuals at Rest provides an expectation that their MI signals will exhibit a reduction within that range, signifying a certain level of connection between these states.

Additional rules as the one described above may describe the connection between the states, as some subjects in specific frequency ranges demonstrated larger differences. The rule described above includes one of multiple rules that are capable of being derived to establish links between the Rest and MI states. Increasing the number of rules will contribute to a comprehensive understanding of MI signals, forming a foundation for substituting the need for MI by having access to the subjects' Rest signals, as described herein. Additional rules may be derived from published works and preliminary results, which demonstrate correlations for multiple subjects based on features. The introduction of more basic measures is likely to yield anticipated consistent observations. As an alternative example, data may be used that adheres to a smaller set of guiding rules while discarding the rest. The discarded data can be considered as an unused cluster.

### Performance Evaluation.

Quantification rules that satisfy two criteria-applying to at least 70% (assuming 30% are BCI-illiterate (Kübler et al., 2004)) of data points and demonstrating statistical significance-were incorporated into the Rest-MI quantifier. Regarding the Rest-Rest similarity metric, there was no performance evaluation to assess, but it is evaluated when implemented in Example #2.

### Results.

The present disclosure relates to development of the Rest-MI quantifier, comprising a set of rules that explain expected MI characteristics based on Rest data. The present disclosure further relates to the establishment of a Rest-Rest similarity mapping mechanism to cluster data and match new data points with the appropriate cluster.

### Example #2: Incorporate Rest Knowledge into an MI-EEG Modeling Framework to Minimize End-User Training Requirements

After the proper quantification rules to link the two states were identified in example #1, such rules guide the work of example 2. example 2 creates a piece-wise classification strategy comprising multiple clusters of data. Inside the clusters, six types of classification methodologies are developed, categorized into basic (such as no need for testing MI data, only Rest), semi-adaptive (such as need for unlabeled testing MI data with Rest), and adaptive (such as need for labeled testing MI data with Rest). The adaptive nature allows the system to continually improve with incoming MI data. example 2 fights nonstationarities (such as differences in signal characteristics) using pre-trained models infused with new subject's Rest data. This proved effective and resulted in satisfactory overall decoding accuracies. example 2 defines satisfactory performance as 75% accuracy for at least 70% of the subjects where 30% of the subjects are BCI-illiterate (Kübler et al., 2004).

Example 2 involves satisfactory accuracy values being consistently attained. If not consistently attained, subjects for whom their MI can be effectively substituted by Rest will still be sufficient to perform methods described herein within their clusters. As an alternative, the amount of MI data required may be minimized such as in semi-adaptive and adaptive frameworks.

### Research Design.

### Data Used and Preprocessing.

As an example, the same data from example #1 was utilized. In example #1, the training data were clustered based on Rest. Each cluster contained both Rest and MI data, referred to as “training data” in FIG. 8. Subsequently, a new Rest testing data point was input into the Rest-Rest similarity function to assign it to a cluster, with the possibility for MI to be included in the cluster (for adaptive models). These are labeled as “testing data” on the figure. Each new testing point matched to a cluster was aligned together via outlier removal, centering (based on Euclidean space), and normalization.

The primary focus of example 2 is on the processing steps that occur within the cluster. Given that no MI is taken for the basic model, the MI data for the testing point was generated synthetically using data from the respective cluster.

### Synthetic MI Data Generation.

Synthetic data generation for the testing point was performed on the training data available in the cluster. It was performed by employing three, example neural network-based (NN-based) methods including Generative Adversarial Networks (GAN), Generative Pre-trained Transformers (GPT), and Variational Auto-Encoders (VAE). GANs employ a dynamic generator-discriminator setup, where a simultaneous and interconnected loss function is employed between the two, resulting in synthetic data generated by the generator that is practically indistinguishable from real data. GPTs are adept at generating data by drawing on historical information ingrained during model training. By providing specific seed information containing pre-defined conditions. And lastly, VAEs operate on an encoder-decoder setup linked through a network, where the data is represented in a latent space. This setup is regulated by minimizing a loss function incorporating a reconstruction loss and a regularization term. The former compels the generated data to resemble the original data, while the latter enforces the latent space to exhibit specific desirable properties.

Each method possesses a unique structural framework, providing varied approaches to integrate specific conditions into the generation process. These frameworks were utilized to incorporate conditions derived from the Rest-MI quantifier. The data generation process was based on the training MI, which already shares common characteristics with testing MI as they fall into the same cluster. This process was refined by regularization with the expected characteristics of testing MI obtained from the quantifier. This ensured that the generated data closely adhered to the inherent patterns and characteristics observed within the MI signals of the respective clusters, thereby producing a tailored and highly realistic synthetic dataset for the testing data point.

Basic Model with Synthetic MI.

### CSP-LDA.

CSP-LDA is a widely recognized and effective approach in EEG-MI signal processing.

### Convolutional NN (CNN)-Long Short-Term Memory (LSTM).

This modern configuration leverages the convolutional layer's ability to handle spatial features in sequential data, while the LSTM layer accommodates the inherent time dependency present in time-series EEG data.

Basic Model without Synthetic MI.

### Regularized CSP-LDA Pipeline.

The covariance matrices fed to the CSP algorithm underwent a transformation to adhere to the rules specified by the Rest-MI quantifier. Multiple versions of these modified covariance matrices were generated, with each focused on maximizing a different rule individually. After that, two classification strategies were employed. First, individual CSP-LDA pipelines were constructed for each version of the data (covariance matrices), and the final decision was determined through a majority voting mechanism. The second strategy involved a singular CSP-LDA pipeline that utilized the combined data to yield optimal results.

### Regularized CNN-LSTM Pipeline.

The loss function of the CNN-LSTM pipeline was adjusted to incorporate a criterion. This criterion aimed to maximize the similarity term, quantifying the resemblance between the neural network layers' parameters from the Rest-MI quantifier and those of the current network. This introduced regularization to guide the solution toward a configuration more closely aligned with the expectations outlined by the quantifier.

Semi-Adaptive Model with Synthetic MI.

The semi-adaptive paradigm refers includes unlabeled testing data gradually introduced to a model, simulating a real-life application. Despite lacking labels (such as which tasks were performed to generate these signals), these data points were valuable, and the following description outlines how they were utilized to continuously improve performance over time. This step essentially involved revisiting the model-building processes outlined above and incorporating the unlabeled data. This was conducted in two examples. In a first example, labels for the unlabeled data were generated by comparing it to the expected MI characteristics as obtained from the quantifier. Then, it was added to the pool of data fed into both pipelines. The second example included adding the unlabeled data to both pipelines and implementing a semi-supervised version for each, to automatically predict its labels and use it in the classification task. The classification pipeline can be either CSP-LDA, or CNN-LSTM.

Semi-Adaptive Model without Synthetic MI.

Instead of using synthetic data, the training data will be regularized as described in the above disclosure. Two example methods were used to utilize unlabeled information. In the first example method, the label for the data was predicted from the quantifier, and it was combined with the original data before being fed into the pipelines. Then, this data point was assigned a higher weight within the pipelines. In the second example method, the data was directly added to the pipelines, and the semi-supervised versions of each pipeline were employed. The classification pipeline can be either: CSP-LDA, or CNN-LSTM.

### Adaptive Model Construction.

The adaptive paradigm includes a scenario in which labeled testing data is incrementally introduced to the model, simulating a real-life system with feedback.

### Regularized Weighted CSP-LDA and CNN-LSTM.

Both pipelines were implemented, leveraging a combination of the following: 1) labeled testing point(s), and 2) the synthetic data or training data regularized based on the quantifier metrics. To maximize the benefit of the MI testing data points, one of two changes was implemented. First, the objective function of each pipeline was regularized towards these data points to enhance confidence in the decisions made for them, or these testing data points were assigned higher weights than the original data points.

### TL-CNN-LSTM.

In this transfer learning (TL) framework for neural network (NN) models, a two-step process was employed. First, a model (Model A) was constructed based on old data, which may be the original cluster's data or synthetic data. Subsequently, another model (Model B) was developed using the available labeled testing points. Next, the first few layers of Model A, which served as general feature extractors, were fixed or “frozen.” These frozen layers were then transferred to Model B, where the specific node parameters were adjusted and fine-tuned according to the characteristics of the given data point. This approach was effective when the data used in model B was limited, which was the case with real-life implementation of any BCI model.

### NF-TL-CNN-LSTM.

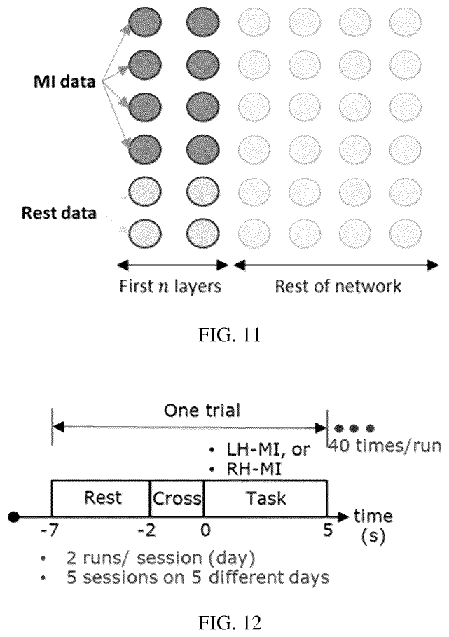

This approach refers to a modified version of a traditional TL framework where in addition to layer freezing, Node Freezing (NF) was introduced to infuse Rest knowledge into the TL pipeline. This method is novel according to the present disclosure and has not been seen in the literature, as shown in FIG. 11.

The primary addition to traditional TL involved introducing several nodes in both models A and B, described herein, which are specific to Rest features. The parameters for these “Rest nodes” were not frozen and transferred from model A to model B. Instead, their weights were recalculated based on the Rest signals from the testing data point. This model surpasses traditional TL methods by having real-time guiding neurons that are most likely to have a greater influence on other MI nodes in the network and provide better performance.

### Complement it Using Synthetic Data.

Labeled testing data was used to generate synthetic data and was complemented through the application of the same three methods (GAN, GPT, and VAE). Subsequently, a testing process was conducted using both pipelines: CSP-LDA and CNN-LSTM.

Preliminary results. The essence of example 2 is that the Rest state can effectively simulate MI data for testing. To support this hypothesis, the following work was conducted: a simple synthetic MI data generation unit was developed based on linear regression. This unit took Rest data and generated MI data for both tasks, built using the data from (BCI Competition IV, 2004). The unit was constructed by regressing one MI channel with all channels of Rest. Therefore, N_{ch} linear regression models are built for a single MI channel. The final value for the MI channel was determined as a weighted average of all values obtained from the various channels, with increased weights assigned to the channels in the hemisphere corresponding to the MI task (left hand-left hemisphere, right hand-right hemisphere.) Regression took place on the Fourier series parameters of the signal (amplitude, frequency, and phase), and then the MI signals were constructed based on these parameters.

This model was applied to other subjects that share common Rest characteristics (in terms of PSD values), imitating two data points in the same cluster without having the quantifier adjust the data generation process. Then, the synthetic data were compared with the real data of the testing subject. One cluster of subjects was considered composed of subjects 1, 2, 3, 6, 7 and 8. The results are shown in Table IX below.

TABLE IX
THE NORMALIZED DISTANCE (ND) OF DTW
ALGORITHM BETWEEN SYNTHETIC AND REAL
MI DATA, BASED ON FZ CHANNEL.

| Subject combination | DTW-ND (LH) | DTW-ND (RH) |
|---|---|---|
| 1-2 | 0.81 | 0.17 |
| 1-3 | 2.49 | 0.99 |
| 1-6 | 2.57 | 0.75 |
| 1-7 | 0.37 | 0.46 |
| 1-8 | 0.28 | 2.86 |
| 2-3 | 1.3 | 0.35 |
| 2-6 | 5.58 | 0.11 |
| 2-7 | 2.90 | 2.91 |
| 2-8 | 0.53 | 3.12 |
| 3-6 | 0.67 | 0.27 |
| 3-7 | 2.86 | 0.61 |
| 3-8 | 0.41 | 3.16 |
| 3-9 | 3.74 | 0.62 |
| 6-7 | 0.47 | 0.47 |
| 6-8 | 0.16 | 2.69 |
| 7-8 | 0.21 | 2.44 |

Table IX presents the normalized distance computed using the DTW algorithm, chosen for its superiority in considering temporal shifts between signals compared to alternative similarity metrics like mean square error. Lower values signify higher similarity, and given the normalization of data, small values (below 1) generally indicate a significant degree of similarity. It is observed that a number of subjects yielded distances below 1. The results in Table IX provide support for utilizing neural network-based synthetic data generators, coupled with precise quantification rules for reducing the need for MI data from the testing subject. Methods involving more complex regression models and quantifier rules to adjust the generated data may be employed to improve results shown in Table IX.

### Results.

Example 2 shows at least two outcomes. example 2 identifies which methods can offer sufficient value in substituting the necessity for conducting MI-EEG data collection sessions by leveraging Rest EEG and determine the extent to which these methods can reduce the need for MI data. example 2 also evaluates the effectiveness of such methods in developing adaptive paradigms that utilize incoming testing data points and enhance their performance over time.

### Example #3: Examine ALS Effects on EEG Signals and Adapt Models from Non-ALS Individuals for Effective Use in an ALS Group

Traditionally, proof-of-concept studies in research often involve individuals without disabilities or neural conditions, raising concerns about the applicability of developed models to real-world users. The present disclosure deviates from this approach by examining EEG differences between two groups: those with and without ALS, using this contrast to validate previous Examples 1 and 2. example 3 relates to quantifying EEG signal differences between the two groups and developing a transition mechanism for non-ALS models to adapt to ALS individuals. This establishes multiple layers of validation for the disclosure in Examples 1 and 2, determining whether addressing the distinctive characteristics of end users' signals should be a post-processing step or integrated into the system design from the outset. example 3 is directed towards the degree and nature of MI-EEG signals between two groups of individuals to inform building EEG classification models tailored to specific populations.

### Research Design.

### Sample Size and Demographics.

As an example, a preliminary estimation suggests a required data size of 30 ALS participants and 60 individuals without neural conditions. This estimation is based on a preliminary power analysis employing a two-tail t-test, Type 1 and Type 2 errors set at 0.05, an effect size of 0.82, and an allocation ratio of 0.5, maintaining a power level of 0.9520. The chosen effect size is linked to motor neuron degeneration associated with ALS and its impact on the ability to produce MI signals. Various evidence-based effect sizes and sample sizes may be used.

Due to challenges in recruiting the ALS group, their age and gender distribution will define the demographics of the comparison group, aiming for an even split between both genders and an age group of 18 years old with no upper limit. This ensures matching demographics for both groups, allowing the cancellation of age and gender effects on signals. Consequently, the power analysis for quantifying differences between the two groups remains valid. Regarding the use of this data to validate the previous aims, the effect of age (Al Zoubi et al., 2018; Schott, 2012) and gender (Butler et al., 2006) on EEG characteristics, the data will undergo a stratified data analysis strategy, considering permutations of 10-year age groups and gender groups.

Collecting data from a non-ALS group, instead of opting for other published datasets beyond those used in developing the models, may be done because there are not enough data points in published datasets to meet required numbers. Also, no available datasets feature multiple sessions with cue-free Rest periods that match the older age groups expected in the ALS group.

### Data Acquisition.

Data will be collected using DSI-7 (Wearable Sensing, 2024). The electrode caps were specifically designed according to methods of the present disclosure to accommodate a large number of electrodes over the motor cortex area while ensuring ease of use. Such caps include at least six electrodes covering the entire motor cortex area and at least two electrodes serving as EOG sensors. This model was selected for its pain-free and rapid setup, requiring less than 1 minute to set up.

Every participant will perform five sessions over five days in each the participant will be asked to perform 20 trials of right-hand MI and 20 left-hand MI (random order), in addition to Rest periods. Each of the segments will last for three seconds. The protocol is shown in FIG. 12.

The trials will be divided into two runs per day, separated by a 10-minute break. In addition, there will be a 2-minute recording at the beginning without performing any tasks with both eyes open and eyes closed to get baseline Rest activity at the beginning of each session.

### Recruitment.

Subjects will have limb-onset ALS, characterized by moderate symptoms in the early stages. Participants must have received a diagnosis within the last two years to ensure uniformity in disease progression stages.

### Data Analysis:

### Difference Between Both Groups' Characteristics.

The data for both groups will be analyzed in a blind-analysis fashion to mitigate bias. All data will be processed after they are stored in the same format without any distinctive properties that would reveal their original group. This process will be handled by one person recruited for this purpose, and the identity of the data will be revealed to the researcher analyzing the data once the results are finalized. The differences will be based on strength features (PSD in small frequency groups), and they will collectively serve as the foundation for statistical comparisons.

### Transition Model.

The present disclosure relates to a transition model becomes necessary. This involves reconstructing the Rest-MI quantifier using the ALS data and revisiting the work in the previous Examples 1 and 2. The collected data will be divided into two segments: one for the model reconstruction and the remaining portion for testing its effectiveness.

### Model Validation.

The models from example 2 will be validated using the collected data from the non-ALS group. The data from the ALS group will validate the model according to the validity of the above described disclosure.

### Performance Evaluation.

Testing the working hypothesis and quantifying the difference in EEG characteristics for both classes (Rest and MI) constitutes the success of the comparison. In the transition phase, establishing the validity of at least one functional model for the ALS group is deemed successful. Lastly, validating the models (attaining a minimum of 75% decoding accuracies for at least 70% of data points) for both groups represents success in this aspect.

Example 3 includes at least several objectives including quantifying the difference in MI EEG signal characteristics between the two groups, quantifying the needed modifications to have non-ALS data used for building ALS models, and demonstrating the validity of the models developed in previous examples.

The present disclosure relates to systems and methods that determine whether: (i) similar Rest features yield similar MI characteristics, and thus (ii) subjects can be grouped based on their Rest signals, and the ML classifiers from one subject can be reused for other subjects with similar Rest characteristics. As a further non-limiting example, a dataset was used which included: BCI competition IV 2a [1] (9 subjects, 72 left hand MI, 72 right hand MI, 1 Rest period) (See “BCI Competition IV.” Accessed: Aug. 1, 2022. [Online]. Available: https://www.bbci.de/competition/iv/). Preprocessing involved bandpass filtration (6-35 Hz) and the utilization of Electrooculogram (EOG) electrodes for isolating eye artifacts through Independent Component Analysis (ICA). For the Rest state, the features utilized are Power Spectral Density (PSD) in the Alpha band (6-11 Hz) at C3 and C4 channels. In the case of MI data, the top 2 Common Spatial Filters (CSPs) were used.

For (i), subjects were divided into 3 groups (3 subjects each), based on the absolute difference in their peak Rest PSD values across both hemispheres. Then, MI classifiers were built using Linear Discriminant Analysis (LDA) for each subject independently to explore potential similarities in the resulting MI distributions (and classifier parameters) among subjects within the same cluster. For (ii), during each testing phase, one subject out of the three was selected for testing, while the other two were used to build separate LDA classifiers. A multiple regression model was then trained using the Rest features (PSD values) and LDA classifier parameters of these two subjects. This model was applied to the testing subject's Rest data to output a constructed classifier for this subject without utilizing any of its MI data.

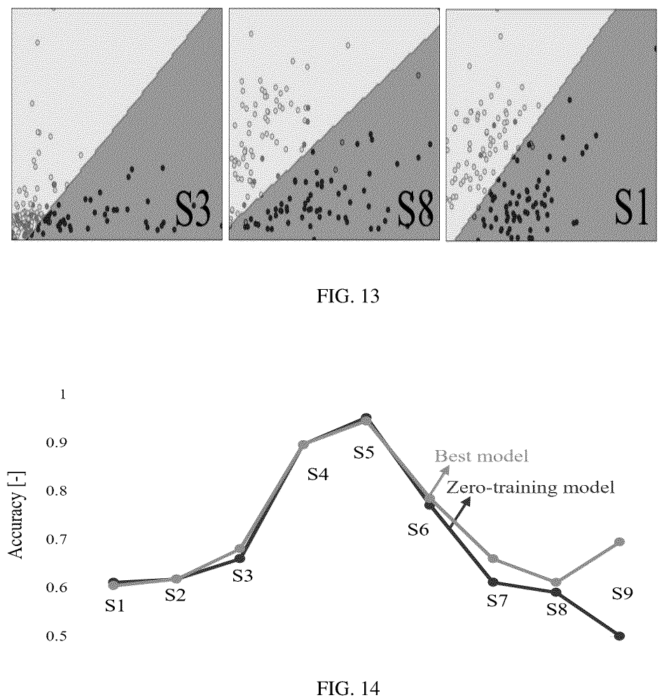

For (i), the resulting MI distributions and decision boundaries for one of the clusters are shown in FIG. 13. This shows that similar Rest leads to similar MI distributions.

For (ii), the accuracies achieved using constructed models (zero training) along with subject-specific models (full training) are shown in FIG. 14. The results indicate close similarities, all performing above chance level.

The results support using Rest EEG and pre-trained MI models to eliminate the need for training data to build MI decoding models, offering a practical foundation for developing EEG-driven biomedical technologies.

Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be or comprise a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described techniques. References in the claims to an element in the singular is not intended to mean “one and only” unless explicitly so stated, but rather “one or more.” All structural and functional equivalents to the elements of the above-described example embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are example only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A method comprising:
extracting, from a data source based on a plurality of users, a motor imagery (MI) task-EEG signals dictionary and rest-EEG signals;
generating, from at least the MI task-EEG signals dictionary and the rest-EEG signals, a confusion matrix that represents at least a contrast between the EEG signals associated with different MI tasks for one or more of the plurality of users;
clustering the confusion matrix based at least on one or more values of the rest-EEG signals;
selecting a representative matrix based on the clustering;
receiving short segments of rest-EEG associated with a target user;
identifying a subcategory based on the received short segments of rest-EEG; and
outputting an indication of a target MI task based on applying the representative confusion matrix to the short segments of rest-EEG based on the identified subcategory.

2. The method of claim 1, wherein the data source comprises EEG data collected while the plurality of users are at rest.

3. The method of claim 1, wherein the data source comprises EEG data collected while the plurality of users perform one or more tasks.

4. The method of claim 1, wherein the data source comprises EEG data collected while the plurality of users are at rest, and wherein the data source comprises EEG data collected while the plurality of users perform one or more tasks.

5. The method of claim 1, wherein the confusion matrix is modeled on at least one rule that provides that a distinction between a rest state and a RH-MI state is that MI signals exhibit x₁% (CI: xₗ_{₁}%-xₕ_{₁}%) higher power in a specific frequency range in left hemisphere channels and x₂% (CI: xₗ_{₂}%-xₕ_{₂}%) lesser power in right hemisphere channels.

6. The method of claim 5, wherein the at least one rule provides that in a LH-MI state, right hemisphere channels exhibit x₃% higher coherence than left hemisphere channels and x₄% (CI: xₗ_{₃}%-xₕ_{₃}%) higher than a rest state.

7. A method comprising:
extracting, from a data source based on a plurality of users, a motor imagery (MI) task-EEG signals dictionary and rest-EEG signals;
generating, from at least the MI task-EEG signals dictionary and the rest-EEG signals, a confusion matrix that represents at least a contrast between the EEG signals associated with different MI tasks for one or more of the plurality of users;
receiving short segments of rest-EEG associated with a target user; and
outputting an indication of a target MI task based on applying the representative confusion matrix to the short segments of rest-EEG.

8. The method of claim 7, wherein the data source comprises EEG data collected while the plurality of users are at rest.

9. The method of claim 7, wherein the data source comprises EEG data collected while the plurality of users perform one or more tasks.

10. The method of claim 7, wherein the data source comprises EEG data collected while the plurality of users are at rest, and wherein the data source comprises EEG data collected while the plurality of users perform one or more tasks.

11. The method of claim 1, wherein the confusion matrix is modeled on at least one rule that provides that a distinction between a rest state and a RH-MI state is that MI signals exhibit x₁% (CI: xₗ_{₁}%-xₕ_{₁}%) higher power in a specific frequency range in left hemisphere channels and x₂% (CI: xₗ_{₂}%-xₕ_{₂}%) lesser power in right hemisphere channels.

12. The method of claim 11, wherein the at least one rule provides that in a LH-MI state, right hemisphere channels exhibit x₃% higher coherence than left hemisphere channels and x₄% (CI: xₗ_{₃}%-xₕ_{₃}%) higher than a rest state.

13. A method for adapting models from a first group of subjects to be used with a second group of subjects, the method comprising:
providing a first sample of individuals in a first group of subjects;
providing a second sample of individuals in a second group of subjects;
providing an apparatus for collecting data from the first and second samples, wherein the apparatus is configured to receive one or more neural signals from the individuals in the first and second samples;
developing a transition model based at least on the first and second samples, wherein the transition model represents at least a contrast between EEG signals associated with different MI tasks for one or more of the subjects;
receiving short segments of rest-EEG associated with a target user; and
outputting an indication of a target MI task based on applying the transition model to the short segments of rest-EEG.

14. The method of claim 13, wherein the first group of subjects does not exhibit a neural disease.

15. The method of claim 13, wherein the second group of subjects exhibits a neural disease.

16. The method of claim 15, wherein the neural disease is amyotrophic lateral sclerosis (ALS).

17. The method of claim 13, wherein the apparatus comprises a cap configured to be worn on the head of an individual.

18. The method of claim 17, wherein the cap comprises at least six electrodes covering a majority of the motor cortex area of the individual and at least two electrodes serving as electrooculography (EOG) sensors.

19. The method of claim 13, wherein data is collected during right-hand MI, left-hand MI, and rest periods.

20. The method of claim 13, wherein the data processing analyzes strength features and serves as the foundation for statistical comparisons between the first group of subjects and the second group of subjects.
